SEQUENCE LISTING (15 sequences)

SEQ ID NO: 1 - RNA, 2 nt, Artificial Sequence
  binding site
uh

SEQ ID NO: 2 - RNA, 6 nt, Artificial Sequence
  misc_feature 3: n = A,U,C or G
rynguc

SEQ ID NO: 3 - DNA, 2 bp, Artificial Sequence
  binding site
ry

SEQ ID NO: 4 - protein, 9 aa, Artificial Sequence
  TAT peptide
RKKRRQRRR

SEQ ID NO: 5 - protein, 9 aa, Artificial Sequence
  TAT peptide
RKKRRQRRR

SEQ ID NO: 6 - protein, 7 aa, Artificial Sequence
  Arg7
RRRRRRR

SEQ ID NO: 7 - protein, 9 aa, Artificial Sequence
  Arg9
RRRRRRRRR

SEQ ID NO: 8 - protein, 7 aa, Artificial Sequence
  Antennapedia Heptapeptide
RRMKWKK

SEQ ID NO: 9 - protein, 17 aa, Artificial Sequence
  Antennapedia Leader peptide
KKWKMRRNQFWVKVQRG

SEQ ID NO: 10 - protein, 9 aa, Artificial Sequence
  TAT 2
RKKRRHRRR

SEQ ID NO: 11 - protein, 10 aa, Artificial Sequence
  TAT 3
RKKRRQHRRR

SEQ ID NO: 12 - protein, 10 aa, Artificial Sequence
  TAT 4
RRKRRQHRRR

SEQ ID NO: 13 - protein, 15 aa, Artificial Sequence
  TAT 5
RKKRRQRRRHRRKKR

SEQ ID NO: 14 - protein, 9 aa, Artificial Sequence
  TAT peptide
RKKRRQRRR

SEQ ID NO: 15 - protein, 15 aa, Artificial Sequence
  dTAT
RKKRRQRRRHRRKKR

(19) United States
(12) Patent Application Publication
(10) Pub. No.: US 2011/0287547 A1
(43) Pub. Date: Nov. 24, 2011

(54) NUCLEIC ACID DELIVERY COMPOSITIONS AND METHODS

(76) Inventor: Cory J. Berkland, Lawrence, KS (US)

(21) Appl. No.: 13/115,763

(22) Filed: May 25, 2011

Related U.S. Application Data
(63) Continuation of application No. PCT/US09/65886, filed on Nov. 25, 2009.
(60) Provisional application No. 61/118,060, filed on Nov. 26, 2008.

(51) Int. Cl. C12N 15/63

(52) U.S. Cl. 435/455; 435/320.1

(57) ABSTRACT

Complexes comprising a cationic polymer, a nucleic acid and a metal ion are provided. In some embodiments, a complex may be used as a means for delivering nucleic acid to a cell. In some embodiments, a complex may be used as part of a gene therapy. Methods of making a complex comprising a cationic polymer, a nucleic acid, and a metal ion are also described. Methods of condensing a polyplex comprising a cationic polymer and a nucleic acid are also provided.

### CROSS-REFERENCE TO RELATED APPLICATIONS

This application is a continuation of International Application No. PCT/US09/65886, filed Nov. 25, 2009, which claims the benefit of U.S. Provisional Patent Application Ser. No. 61/118,060, filed Nov. 26, 2008, the entire disclosures of which are incorporated by reference.

### BACKGROUND

The present disclosure, according to certain embodiments, generally relates to nucleic acid delivery. In particular, in some embodiments, the present disclosure provides compositions and methods for delivering nucleic acid to a cell using a complex comprising a cationic polymer, a nucleic acid and a metal ion.

Gene therapy, the therapeutic manipulation of gene expression, has been proposed as a rational new treatment option for a multitude of conditions, including both inherited and infectious diseases and cancer. Gene therapy can also be used to promote the localized healing of injured tissue. More recently, the manipulation of gene expression has been accomplished through interfering RNA technologies. Each of these indications requires specific therapeutic targets and discrete delivery strategies.

Efficient expression of genetic material for therapeutic indications remains, primarily, a delivery problem. DNA must be packaged in a way that enables it to avoid degradation, enter target cells, escape into the cytoplasm, and be delivered into the cell nucleus for expression. Similar barriers impede the delivery of interfering RNA into cells.

Currently, the introduction of genes into cells relies primarily on either viral vectors or non-viral vectors. The most successful gene therapy strategies to-date employ the use of viral vectors. Viral vectors commonly used in this setting are adenoviruses, adeno-associated viruses, retroviruses, and the herpes virus. But these vectors generally suffer from problems associated with immunogenecity, cytotoxicity, and mutagenesis.

Non-viral vectors are typically thought to be a safer alternative to viral vectors. Commonly-used non-viral vectors include polymers, liposomes, peptides, and polysaccharides. These materials are also being explored to deliver RNA-based therapeutics. Unfortunately, the use of non-viral vectors in gene therapy settings is generally inefficient and toxic at times. For instance, recent studies reveal that current polymer/DNA complexes, such as polyethylenimine/DNA, that are effective at DNA delivery often suffer from high toxicity. Additionally, although biodegradable polymeric gene vectors containing peptides or polysaccharides are less toxic than viral vectors and are fully biodegradable in vivo, they are often unable to regulate (e.g. enhance or diminish) gene expression levels and persistence. It is well recognized that there is an urgent need for non-toxic and efficient nucleic acid delivery methods to fully exploit the current potential of these therapies in molecular medicine.

### SUMMARY

The present disclosure provides complexes comprising a cationic polymer, a nucleic acid and a metal ion. In some embodiments, a complex of the present disclosure can be used as a means for delivering nucleic acid to a cell. In some embodiments, the complexes of the present disclosure may be used as part of a gene therapy. The methods of making a complex comprising a cationic polymer, a nucleic acid, and a metal ion are also described. Furthermore, methods of condensing a polyplex comprising a cationic polymer and a nucleic acid are also provided.

### DRAWINGS

For a more complete understanding of the present disclosure, and the advantages thereof, reference is now made to the following descriptions taken in conjunction with the accompanying drawings, in which:

FIG. 1 is a graph showing the cytotoxicity test of both polyethylenimine (PEI) and TAT.

FIG. 2 is a graph showing the cytotoxicity test of CaCl₂.

FIG. 3 is a graph showing the effect of CaCl₂ concentration on particle size of PEI complexes (N/P 5, N/P 10) and TAT-Ca complexes.

FIG. 4 is a graph showing the effect of CaCl₂ concentration on charge of PEI complexes (N/P 5, N/P 10) and TAT-Ca complexes.

FIG. 5 is a graph showing the transfection efficiency of both TAT and PEI polyplexes with varying concentrations of CaCl₂.

FIG. 6 is a graph showing the transfection efficiency of both TAT and PEI complexes with and without 0.3 M CaCl₂.

FIG. 7 is a graph showing the transfection efficiency of both TAT-Ca and PEI complexes.

FIG. 8 is a graph showing the effect of CaCl₂ concentration on both the TAT and PEI complexes.

FIG. 9 is a graph showing the unpackaging of both TAT and PEI complexes as a function of heparin.

FIG. 10 is a graph showing the condensation of both TAT and PEI complexes by using a TNBS assay.

FIG. 11 is a graph showing the Luciferase gene silencing efficiency mediated by TAT-Ca and by PEI siRNA (30 nM of siRNA) complexes in A549 cells.

FIG. 12A is a gel electrophoresis study showing pDNA-CaCl₂ complexes at varying CaCl₂ concentrations; 1=0.1M, 2=0.2M, 3=0.3M, 4=0.4M, 5=0.5M, 6=0.6M, 7=0.7M, 8=0.8M, 9=0.9M, 10=1.0M, 11=3M, 12=5M, 13=7M of CaCl₂.

FIG. 12B is a gel electrophoresis study showing TAT-pDNA complexes with 0.3M CaCl₂ at varying N/P ratios.

FIG. 12C is a gel electrophoresis study showing TAT-pDNA complexes at varying N/P ratios.

FIG. 12D is a gel electrophoresis study showing TAT-CaCl₂ (N/P 25) complexes at varying CaCl₂ concentrations; 1=0 mM, 2=62.5 mM, 3=125 mM, 4=250 mM, 5=300 mM, 7=400 mM, 8=500 mM, and 10=2M of CaCl₂.

FIG. 12E is a gel electrophoresis study showing branched PEI-pDNA complexes.

FIG. 12F is a gel electrophoresis study showing linear PEI-pDNA complexes.

FIG. 13 is a graph showing the transfection efficiency of both TAT and PEI complexes in A549 cells in the absence or presence of 10% FBS.

FIG. 14A is a graph showing the stability of particle size as a function of concentration and time in the absence of serum.

FIG. 14B is a graph showing the stability of particle size as a function of concentration and time in the presence of 10% FBS.

FIG. 15A is a graph showing the condensation of pDNA (pGL3) by PLL at varying molecular weights.

FIG. 15B is a graph showing the condensation of pDNA (pGL3) by PEI at varying molecular weights.

FIG. 16 is a graph showing the effect of CaCl₂ concentration on the condensation of pDNA (pGL3) by polyamine.

FIG. 17A is a graph showing the effect of CaCl₂ concentration on the gene expression of pDNA/PLL complexes.

FIG. 17B is a graph showing the effect of CaCl₂ concentration on the gene expression of pDNA/PEI complexes.

FIG. 17C is a graph showing the effect of CaCl₂ concentration on the gene expression of pDNA/polyamine.

FIG. 18A is a graph showing the results of a cytotoxicity assay.

FIG. 18B is a graph showing the results of a cytotoxicity assay.

FIG. 18C is a graph showing the results of a cytotoxicity assay.

FIG. 18D is a graph showing the results of a cytotoxicity assay.

FIG. 18E is a graph showing the results of a cytotoxicity assay.

FIG. 19A is a graph showing the results of siRNA delivery.

FIG. 19B is a graph showing the results of siRNA delivery.

FIG. 19C is a graph showing the results of siRNA delivery.

FIG. 19D is a graph showing the results of siRNA delivery.

FIG. 19E is a graph showing the results of siRNA delivery.

FIG. 19F is a graph showing the results of siRNA delivery.

FIG. 20 is a graph showing the transfection efficiency of different CPPs-Ca/pDNA (0.3M) and PEI complexes in A549 cells after 2 days.

FIGS. 21A and 21B are graphs showing the stability of CPPs-Ca/pDNA (0.3M) over time in the absence and presence of 10% FBS.

FIG. 22 is a graph showing the cytotoxicity test of both PEI and CPPs.

FIG. 23A is a graph showing the transfection efficiency of a CPP-Ca/pDNA complex (Arg7/pGL3) with different concentrations of CaCl₂ (75, 150, 300 mM).

FIG. 23B is a graph showing the transfection efficiency of a CPP-Ca/pDNA complex (Arg9/pGL3) with different concentrations of CaCl₂ (75, 150, 300 mM).

FIG. 23C is a graph showing the transfection efficiency of a CPP-Ca/pDNA complex (Ahp/pGL3) with different concentrations of CaCl₂ (75, 150, 300 mM).

FIG. 23D is a graph showing the transfection efficiency of a CPP-Ca/pDNA complex (Alp/pGL3) with different concentrations of CaCl₂ (75, 150, 300 mM).

FIG. 24 is a graph showing the transfection efficiency of TAT complexes with varying both the N/P ratios and the concentrations of CaCl₂.

FIG. 25A is a graph showing the transfection efficiency of TAT2 complexes with varying both the N/P ratios and the concentrations of CaCl₂.

FIG. 25B is a graph showing the transfection efficiency of TAT3 complexes with varying both the N/P ratios and the concentrations of CaCl₂

FIG. 25C is a graph showing the transfection efficiency of TAT4 complexes with varying both the N/P ratios and the concentrations of CaCl₂.

FIG. 25D is a graph showing the transfection efficiency of TAT5 complexes with varying both the N/P ratios and the concentrations of CaCl₂.

FIG. 26 is a graph showing the GAPDH gene silencing efficiency mediated by TAT-Ca, Lipofectamine 2000, and Lipofectamine RNAiMAX siRNA (50 nM of siRNA) complexes in HeLa cells.

FIG. 27 is a graph showing the cytotoxicity profiles of PEI, TAT and dTAT for A549-luc-C8 cells. Viability is expressed as a function of polymer concentration. Results are presented as mean±SD (n=3).

FIG. 28A is a graph showing the luciferase knockdown of TAT complexes as a function of N/P ratio condensed with different concentrations of added CaCl₂. The dose was 10 nM of siRNA. Results are presented as mean±SD (n=3).

FIG. 28B is a graph showing the luciferase knockdown of TAT complexes as a function of N/P ratio condensed with different concentrations of added CaCl₂. The dose was 25 nM of siRNA. Results are presented as mean±SD (n=3).

FIG. 28C is a graph showing the luciferase knockdown of TAT complexes as a function of N/P ratio condensed with different concentrations of added CaCl₂. The dose was 50 nM of siRNA. Results are presented as mean±SD (n=3).

FIG. 29A is a graph showing the luciferase knockdown of dTAT complexes as a function of N/P ratio condensed with different concentrations of added CaCl₂. The dose was 10 nM of siRNA. Results are presented as mean±SD (n=3).

FIG. 29B is a graph showing is a graph showing the luciferase knockdown of dTAT complexes as a function of N/P ratio condensed with different concentrations of added CaCl₂. The dose was 25 nM of siRNA. Results are presented as mean±SD (n=3).

FIG. 29C is a graph showing is a graph showing the luciferase knockdown of dTAT complexes as a function of N/P ratio condensed with different concentrations of added CaCl₂. The dose was 50 nM of siRNA. Results are presented as mean±SD (n=3).

FIG. 30 is a graph showing the percentage of GAPDH mRNA expression per tissue relative to the negative control siRNA (n=3).

FIG. 31 is a graph showing the accumulation of GAPDH siRNA per tissue relative to mice given PBS placebo (n=3).

While the present disclosure is susceptible to various modifications and alternative forms, specific example embodiments have been shown in the figures and are described in more detail below. It should be understood, however, that the descriptions of specific example embodiments is not intended to limit the invention to the particular forms disclosed, but on the contrary, this disclosure is to cover all modifications and equivalents as illustrated, in part, by the appended claims.

### DESCRIPTION

The present disclosure, according to certain embodiments, generally relates to nucleic acid delivery. In particular, in some embodiments, the present disclosure provides compositions and methods for delivering nucleic acid to a cell using a complex comprising a cationic polymer, a nucleic acid and a metal ion.

In one embodiment, the present disclosure provides a complex comprising a cationic polymer, a nucleic acid and a metal ion. In some embodiments, a complex of the present disclosure may be used as a delivery vehicle for a nucleic acid to a cell. Furthermore, in some embodiments, a complex of the present disclosure may be used as part of a gene therapy, in which a host cell is transfected with a complex of the present disclosure. One of the many potential advantages of the compositions and methods of the present disclosure is that they may, among other things, provide high and/or sustained gene expression, as well as offer minimal toxicity compared to commonly used gene vectors, such as polyethylenimine. Furthermore, in some embodiments, the methods and compositions of the present disclosure may provide enhanced transfection efficiency when compared to commonly used gene vectors.

As previously mentioned, the present disclosure provides a complex comprising a cationic polymer. Cationic polymers suitable for use in the complexes of the present disclosure generally are positively charged peptides and, in some embodiments, have an amino acid composition containing a high relative abundance of positively charged amino acids, such as lysine or arginine. In some embodiments, a cationic polymer suitable for use in the present disclosure may comprise between about 30% and about 100% cationic amino acids. It is believed that the positive charge of the cationic polymer allows it to interact with the negatively charged phosphate backbone of a nucleic acid through noncovalent, electrostatic interactions. Such electrostatically bound cationic polymer-nucleic acid complexes are referred to herein as “polyplexes.” In some embodiments, cationic polymers suitable for use in the present disclosure are peptides having a molecular weight less than or equal to about 2,000 daltons. In some embodiments, cationic polymers suitable for use in the present disclosure are peptides having a molecular weight less than or equal to about 5,000 daltons. In other embodiments, cationic polymers suitable for use in the present disclosure are peptides having a molecular weight less than or equal to about 10,000 daltons. In other embodiments, cationic polymers suitable for use in the present disclosure are peptides having a molecular weight less than or equal to about 15,000 daltons. In some embodiments, cationic polymers suitable for use in the present disclosure are not part of a larger construct (e.g., not conjugated to other molecules).

In other embodiments, the cationic polymer is a portion of a larger construct that may include a domain to improve polyplex stability, reduce polyplex size, impart function to the polyplex (e.g. targeting), add function to the polyplex (e.g. bioimaging), or similar extensions that would be evident to one skilled in the art. For examples, the cationic polymer, in certain embodiments, may include a targeting moiety that functions to target the complex to a region of interest. Examples of suitable targeting moieties include, but are not limited to, antibody fragments, peptides, aptimers, and small molecules. Any targeting moiety is suitable so long as the cationic polymer is capable of forming a complex. In some embodiments, the targeting moiety may be linked to the cationic polymer through a spacer. Examples of suitable spacers include PEG, peptides formed form repeating hydrophilic amino acids, and the like. Any spacer is suitable so long as the cationic polymer is capable of forming a complex. In some embodiments, the spacer may be linked to a targeting moiety.

In certain embodiments, a cationic polymer suitable for use in the present disclosure may comprise a cell penetrating peptide (CPP). CPPs are short peptides that may facilitate cellular uptake of nucleic acid associated with the peptide through a non-covalent interaction. CPPs suitable for use in the present disclosure typically have an amino acid composition containing a high relative abundance of positively charged amino acids. In certain embodiments, it is desirable for a cationic polymer suitable for use in the present disclosure to comprise a CPP because CPPs are often able to rapidly traverse the membrane of a biological cell. In some embodiments, CPPs suitable for use in the present disclosure have a molecular weight less than or equal to about 5,000 daltons.

One example of a suitable CPP for use in the present disclosure is the trans-activating transcriptional activator (TAT) from Human Immunodeficiency Virus 1 (HIV-1), hereinafter referred to as “HIV-1 TAT.” HIV-1 TAT is a peptide that comprises a protein transduction domain and a nuclear localization sequence. It is believed that peptide sequences derived from protein transduction domains are able to selectively lyse the endosomal membrane in its acidic environment leading to cytoplasmic release. Furthermore, it is believed that the nuclear localization sequence of the HIV-1 TAT peptide is able to facilitate the nuclear transport due to its interaction with the endogenous cytoplasmic-nuclear transport machinery.

In addition to a cationic polymer, a complex of the present disclosure also comprises a nucleic acid. Nucleic acid suitable for use in the present disclosure may be any nucleic acid useful for delivery into a cell (e.g., a bioactive nucleic acid). The term “nucleic acid” as used herein refers to an oligomer or polymer of ribonucleic acid (RNA) or deoxyribonucleic acid (DNA) or mimetics thereof. This term includes nucleic acids composed of naturally-occurring nucleobases, sugars, and covalent internucleoside (backbone) linkages as well as nucleic acids having non-naturally-occurring portions which function similarly. Such modified or substituted nucleic acids are often preferred over native forms because of desirable properties such as, for example, enhanced cellular uptake, enhanced affinity for nucleic acid target, and increased stability in the presence of nucleases. In some embodiments, a nucleic acid may have a structure designed to achieve a well-known mechanism of activity and may include, but is not limited to, siRNA, shRNA, miRNA, a catalytic RNA (ribozyme), a catalytic DNA, an aptazyme or aptamer-binding ribozyme, a regulatable ribozyme, a catalytic oligonucleotide, a nucleozyme, a DNAzyme, a RNA enzyme, a minizyme, a leadzyme, an oligozyme, or an antisense nucleic acid.

Thus, a nucleic acid to be delivered may be a DNA or a RNA molecule, or any modification or combination thereof. In some embodiments, the nucleic acid may contain internucleotide linkages other than phosphodiester bonds, such as phosphorothioate, methylphosphonate, methylphosphodiester, phosphorodithioate, phosphoramidate, phosphotriester, or phosphate ester linkages, resulting in increased stability. Oligonucleotide stability may also be increased by incorporating 3′-deoxythymidine or 2′-substituted nucleotides (substituted with, e.g., alkyl groups) into the oligonucleotides during synthesis or by providing the oligonucleotides as phenylisourea derivatives, or by having other molecules, such as aminoacridine or poly-lysine, linked to the 3′ ends of the oligonucleotides. Modifications of the RNA and/or DNA nucleotides may be present throughout the oligonucleotide or in selected regions of the oligonucleotide, for example, the 5′ and/or 3′ ends. The nucleic acid can be made by any method known in the art, including standard chemical synthesis, ligation of constituent oligonucleotides, and transcription of DNA encoding the oligonucleotides. For example, the oligonucleotides may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. The oligonucleotides also may be produced by expression of all or a part of the target sequence in an appropriate vector.

In one embodiment, the nucleic acid may be an antisense nucleic acid sequence. The antisense sequence is complementary to at least a portion of the 5′ untranslated, 3′ untranslated, or coding sequence. Such antisense nucleic acids must be of sufficient length to specifically interact (hybridize) with a target sequence, but not so long that the nucleic acid is unable to discriminate a single based difference. For example, for specificity the nucleic acid is at least six nucleotides in length. Longer sequences can also be used, depending on efficiency of inhibition, specificity, including absence of cross-reactivity, and the like. The maximum length of the sequence will depend on maintaining its hybridization specificity, which depends in turn on the G-C content of the agent, melting temperature (Tm) and other factors, and can be readily determined by calculation or experiment, for example, stringent conditions for detecting hybridization of nucleic acid molecules as set forth in “Current Protocols in Molecular Biology,” Volume I, Ausubel et al., eds. John Wiley: New York N.Y., pp. 2.10.1-2.10.16, or by utilization of free software such as Osprey (Nucleic Acids Research 32(17):e133) or EMBOSS (http://www.uk.embnet.org/Software/EMBOSS).

In another embodiment, the nucleic acid may be an inhibitory RNA sequence (e.g. siRNA, shRNA, miRNA, etc.). Design of inhibitory RNA molecules is well known in the art and established parameters for their design have been published (Elbashir, et al. EMBO J. 2001; 20: 6877-6888). Similarly, methods of using RNAi-directed gene silencing are known and routinely practiced in the art, including those described in D. M. Dykxhoorn, et al., Nature Reviews 4:457-67 (2003) and J. Soutschek, et al., Nature 432:173-78 (2004). In some embodiments, a target sequence beginning with two AA dinucleotide sequences is preferred because siRNAs with 3′ overhanging UU dinucleotides are the most effective. It is recommended in siRNA design that G residues be avoided in the overhang because of the potential for the siRNA to be cleaved by RNase at single-stranded G residues. Suitable siRNA can be produced by several methods, such as chemical synthesis, in vitro transcription, siRNA expression vectors, and PCR expression cassettes.

In another embodiment, the nucleic acid may be a ribozyme. Design and testing efficacy of ribozymes is well known in the art (Tanaka et al., Biosci Biotechnol Biochem. 2001; 65:1636-1644). It is known that a hammerhead ribozyme requires a 5′ UH 3′ (SEQ ID NO:1) sequence (where H can be A, C, or U) in the target RNA, a hairpin ribozyme requires a 5′ RYNGUC 3′ (SEQ ID NO:2) sequence (where R can be G or A; Y can be C or U; N represents any base), and the DNA-enzyme requires a 5′ RY 3′ (SEQ ID NO:3) sequence (where R can be G or A; Y can be C or U). Based on the foregoing design parameters, a skilled practitioner will be able to design an effective ribozyme either in hammerhead, hairpin, or DNAzyme format. For testing the comparative activity of a given ribozyme, an RNA substrate which contains the common target sequence can be used.

In addition to a cationic polymer and a nucleic acid, a complex of the present disclosure also comprises a metal ion. In general, suitable metal ions should be biocompatible and have a suitable level of toxicity. In certain embodiments, the metal ion may be any metal ion capable of condensing a polyplex comprising a cationic polymer and a nucleic acid, so as to form a complex. Examples of suitable metal ions include divalent metal cations, such as Mg²⁺, Mn²⁺, Ba²⁺, and Ca²⁺.

The amount of metal ion present in a complex of the present disclosure may be tailored to achieve a desired result. For example, the metal ion may be present in an amount that maximizes gene expression (in some applications, gene expression may be a function of metal ion concentration), that minimizes toxicity, that minimizes/condenses the size of the complex (smaller complexes tend to improve delivery, e.g., gene transfection), and/or that optimizes deliverability of the nucleic acid (e.g., using a concentration so the nucleic acid is capable of being released from the cationic polymer once delivered to the cell). In some embodiments, the metal ion concentration is between about 5 and about 1000 mM. In some embodiments, the metal ion concentration is between about 5 and about 800 mM. In some embodiments, the metal ion concentration is between about 5 and about 500 mM. In some embodiments, the metal ion concentration is between about 10 and about 250 mM. However, other embodiments with single metal ions or mixtures of metal ions may have a broader range of concentrations that are able to condense a complex of the present disclosure to nanoscopic dimensions, which may be efficient and/or of low toxicity for nucleic acid transfer to cells.

In some embodiments, a metal ion may be added to a polyplex in an amount sufficient to condense the polyplex, thereby producing a complex with a desired diameter that is smaller than that of the polyplex to which it was added. For example, in some embodiments, a polyplex of the present disclosure may have a diameter greater than approximately 250 nanometers (nm) and, upon the addition of the metal ion, it is condensed so as to form a complex that is smaller in size than the polyplex. In some embodiments, a polyplex of the present disclosure may have a diameter greater than approximately 500 nm and, upon the addition of the metal ion, it is condensed so as to form a complex that is smaller than 500 nm.

The complexes of the present disclosure generally have a diameter of about 500 nm or less. In some embodiments, the complexes of the present invention have a diameter from about 30 nm to about 150 nm. In some embodiments, it may be particularly desirable for a complex of the present disclosure to have a diameter less than 150 nm to facilitate its uptake into a cell.

The complexes of the present disclosure are generally noncytotoxic or minimally cytotoxic. In certain embodiments, the complexes of the present disclosure may have a IC50 (half maximal inhibitory concentration) greater than or equal to about 5 mg/ml. In other embodiments, the complexes of the present disclosure may have a IC50 (half maximal inhibitory concentration) greater than or equal to about 1 mg/ml. In other embodiments, the complexes of the present disclosure may have a IC50 (half maximal inhibitory concentration) greater than or equal to about 500 μg/ml.

To facilitate a better understanding of the present invention, the following examples of certain aspects of some embodiments are given. In no way should the following examples be read to limit, or define, the entire scope of the invention.

### EXAMPLES

### Example 1

Materials. Plasmid DNA encoding firefly luciferase (pGL3, 4.8 kbp) was obtained from Promega (Madison, Wis., USA) and transformed into E. coli (DH5α) (Invitrogen, Carlsbad, Calif.). A single transformed colony picked from an agar plate was cultured in LB Broth Base (Invitrogen) liquid for plasmid DNA preparation. Plasmid DNA (“pDNA”) was purified with Plasmid Giga Kit (5) (Qiagen, Germantown, Md.) following the manufacturer's instructions. All pDNA had purity levels of 1.8 or greater as determined by inspection by UV/Vis (A260/A280). TAT peptide (RKKRRQRRR (SEQ ID NO:4); MW=1338.85 Da) was synthesized in house. Arginine 7 (Arg7), Arginine 9 (Arg9), Antennapedia Heptapeptide (Ahp), Antennapedia Leader peptide (Alp) peptides were obtained from π Proteomics (Huntsville, Ala.). Branched polyethylenimine (PEI, 25 kDa) was obtained from Aldrich (Milwaukee, Wis.). Calcium chloride (CaCl₂.2H₂O) and agarose medium were purchased from Fisher Scientific (Pittsburgh, Pa.). Lipofectamine 2000, and Lipofectamine RNAiMAX transfection reagents were purchased from (Invitrogen).

Human lung carcinoma cell line A549 cells were obtained from the American Type Culture Collection (ATCC, Rockville, Md.). The cell culture medium (Ham's F-12K Nutrient Mixture, Kaighn's modified with L-glutamine) was purchased through Fisher Scientific. Fetal bovine serum (FBS) was purchased from Hyclone. Penicillin-streptomycin was purchased from MB Biomedical, LLC. Trypsin-EDTA was purchased through Gibco. MTS reagent [tetrazolium compound; 3-(4,5-dimethylthiazol-2-yl)-5-(3-carboxymethoxyphenyl)-2-(4-sulfophenyl)-2H-tetrazolium, inner salt] was purchased from Promega.

### Preparation of TAT-Ca/pDNA and TAT-Ca/siRNA Complexes

Particles of nano-sized TAT-Ca complexes were synthesized by rapidly adding and stirring 10 μL of either (0.1 μg/μL) pDNA or (30-50 nM) siRNA to 15 μL (1 μg/μL) of the TAT solution. To this solution, 15 μL of CaCl₂ of known molarity (e.g. 0.3 M) was added and mixed by vigorous pipetting followed by 20-30 minutes incubation at room temperature or at 4° C. prior to use.

Preparation of PEI/pDNA Complexes. Polyethylenimine-DNA Complexes were prepared by adding 10 μl (0.1 μg/μL) of pDNA solution to 15 μL (N/P ratio of 5 or 10) polyethylenimine (PEI) solution drop-wise while stirring. Complexes were incubated at room temperature for 20-30 minutes before dilution 1.7 times (15 μL) with the appropriate buffer (e.g. nuclease-free water or CaCl₂). Complexes were freshly prepared before each individual experiment.

Size and Zeta Potential Measurement. Suspensions containing complexes with TAT or PEI were prepared as described earlier using a pDNA concentration of 0.1 μg/μL. All samples intended for light scattering analyses were prepared using 10 mM Tris buffer, pH 7.4, which was pre-filtered with a 0.22 μm filter to remove any trace particulates. Particle sizes were measured by dynamic light scattering (DLS) using a Brookhaven (Holtsville, N.Y.) instrument equipped with a 9000AT autocorrelator, a 50 mW HeNe laser operating at 532 nm (JDS Uniphase), an EMI 9863 photomultiplier tube, and a BI 200M goniometer. The light scattered at 90° from the incident light was fit to an autocorrelation function using the method of cumulants. Zeta potential measurements were obtained by phase analysis light scattering using a Brookhaven Zeta PALS instrument. The electrophoretic mobility of the samples was determined from the average of 10 cycles of an applied electric field. The zeta potential of complexes was determined from the electrophoretic mobility by means of the Smoluchowski approximation.

Agarose Gel Electrophoresis Assays. The pDNA binding ability of the TAT-Ca/pDNA complexes and PEI/DNA complexes was analyzed by agarose gel electrophoresis. The TAT-Ca/pDNA and PEI/DNA complexes containing 1 μg luciferase reporter gene were prepared as described at various N/P ratios. The N/P ratio refers to the molar ratio of amine groups in the cationic polymer, which represent the positive charges, to phosphate groups in the plasmid DNA, which represent the negative charges. The DNA complex solutions (i.e. 25 μL) at various N/P ratios were diluted by adding 4 μL of 10× Tris-acetate-EDTA (TAE) gel running buffer (Promega) and 4 μL of 100× SYBR Green (Invitrogen) solutions. Six times DNA loading buffer (7 μL) was added to the complex solutions. The mixtures were allowed to incubate at room temperature for 40 minutes to ensure labeling of the DNA with the SYBR Green dye. Thereafter, the complexes were loaded into individual wells of 1% agarose 1× TAE gel buffer, and subjected to electrophoresis at 110 V for 30 minutes. Uncomplexed DNA diluted with an identical volume of solution was used as a control. The resulting DNA migration patterns were revealed using AlphaImager® Imaging System (Alpha Innotech, San Leandro, Calif.).

Cell Culture. Culturing of human epithelial lung cell line A549 was performed according to the protocol provided by the American Type Culture Collection. A549 was grown in F-12K supplemented with 10% v/v FBS and 1% v/v Penicillin/streptomycin at 37° C. in a humidified air atmosphere containing 5% CO₂.

In Vitro Cell Transfection Studies. A549 cells were trypsinized, counted and diluted to a concentration of approximately 80,000 cells/mL. Then 0.1 mL of that dilution was added to each well of a 96-well plate and the cells were incubated in a humidified atmosphere of 5% CO₂ incubator at 37° C. for 24 hours. Immediately before transfection, the cells were washed once with PBS and 100 μl sample (20% of complex to 80% of serum free cell culture medium) was added to each well. Cells were incubated with the complexes for 5 hours. The transfection agent was then removed by aspiration and 100 μL of fresh serum medium was added followed by further incubation. The Luciferase Assay System from Promega was used to determine gene expression following the manufacturer's recommended protocol. The light units were normalized against protein concentration in the cells extracts, which were measured using the Coomassie Plus™ Protein Assay (Thermo Scientific). The transfection results were expressed as Relative Light Units (RLU) per mg of cellular protein.

Cytotoxicity Assay (MTS Assay). Cytotoxicity of the complexes was determined by the CellTiter 96® Aqueous Cell Proliferation Assay (Promega). A549 cells were grown as described in the transfection experiments. Cells were treated with the samples for approximately 24 hours. The media were then removed and replaced with a mixture of 100 μl fresh culture media and 20 μL MTS reagent solution. The cells were incubated for 3 hours at 37° C. in the 5% CO₂ incubator. The absorbance of each well was then measured at 490 nm using a microtiter plate reader (SpectraMax, M25, Molecular Devices Corp., CA) to determine cell viability.

SYBR Green assay of TAT/pDNA and PEI/pDNA Complexes. The degree of pDNA accessibility following complexation with TAT or PEI was assessed by the double-stranded-DNA-binding reagent SYBR Green (Invitrogen). Briefly, 10 μl, (0.1 mg/mL) of pDNA was mixed with 15 μL of TAT or PEI solution, then 15 μL deionized water or metal solution was added. Complexes were then allowed to form for 30 minutes at room temperature prior to use. After incubation, 120 μL deionized water and 160 μL 10× SYBR Green solutions were added. And then 80 μL of each sample was added to triplicate wells of 96-well cell culture plate. The plate was measured by a fluorescence plate reader (SpectraMax M5; Ex., 250 nm; Em, 520 nm).

TNBS assay of TAT/pDNA and PEI/pDNA Complexes. The degree of free amine group of TAT and PEI accessibility following complexation with pDNA was measured by a colorimetric assay with 2,4,6-trinitro-benzenesulphonic acid (TNBS) as an assay reagent (Pierce). Briefly, 10 μL of complex solution was added to 190 μL deionized water and then 200 μL of 0.02% TNBS solution in 0.1 M sodium bicarbonate buffer (pH 8.5) was added. The solution was rapidly mixed. After incubation at 37° C. for 2 hours, 80 μL of sample was added to triplicate wells of 96-well cell culture plate. The absorption at 335 nm was determined on a plate reader (SpectraMax M5).

Results

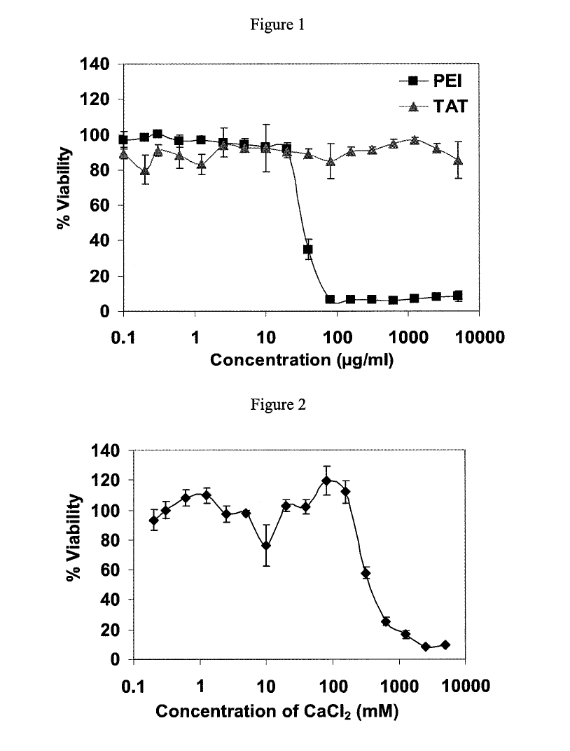

TAT revealed no evidence of cytotoxic effects and cells maintained a high viability, while branched PEI and CaCl₂ induced cytotoxicity (IC₅₀˜35 μg/mL and ˜0.21 M, respectively) (FIGS. 1 and 2).

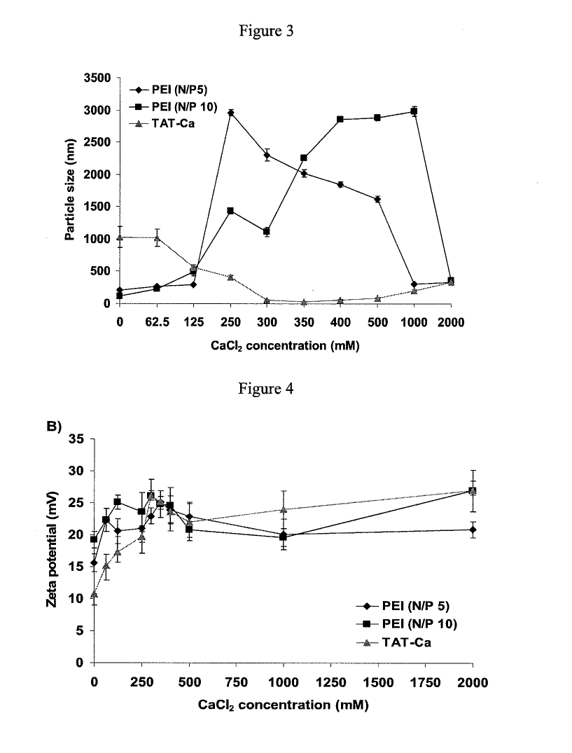

Calcium addition to TAT/pDNA complexes induced a substantial decrease in the particle size compared with those of PEI/pDNA complexes which showed some increase in particle sizes (FIG. 3).

A slight increase in zeta potentials were observed with increasing concentrations of CaCl₂. The values ranged from 11 to 27 mV (FIG. 4).

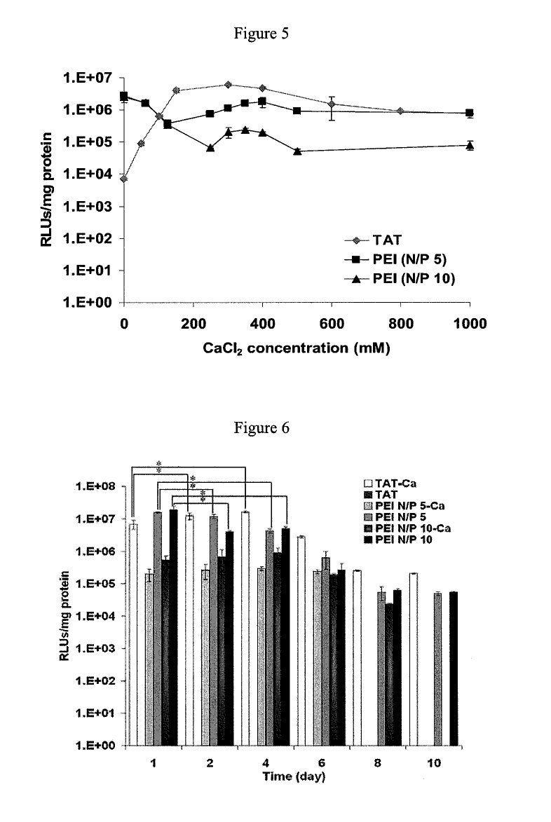

Luciferase gene expression complexed with TAT was evaluated 1 day after transfection as a function of the concentration of CaCl₂. TAT complexes showed a higher level of gene expression at 0.3 M CaCl₂ compared with those of PEI, which had high transfection efficiency in the absence of CaCl₂ (FIG. 5).

The level of gene expression induced by TAT-Ca/pDNA complexes was similar to the transfection efficiency of branched PEI and increased over the first four days, whereas the gene expression of PEI/pDNA complexes showed a marked decrease during the same time frame (FIG. 6).

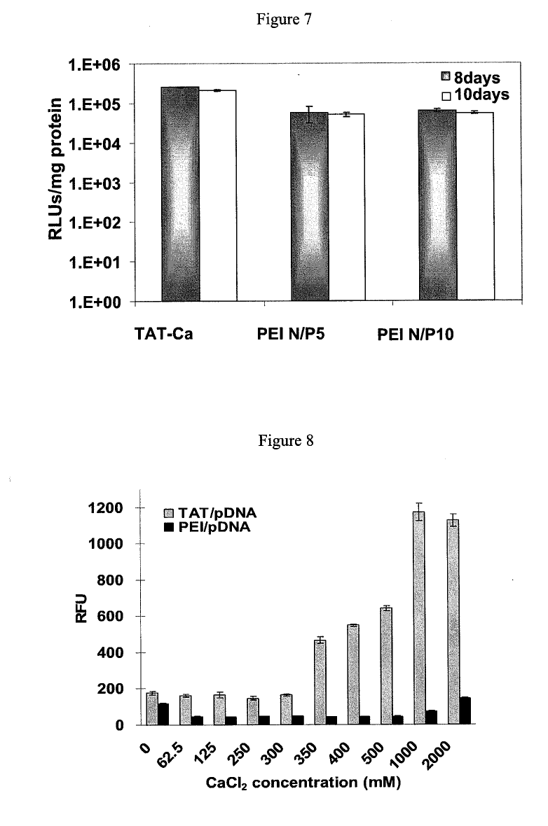

The gene expression was detectable for at least 10 days and TAT-Ca/pDNA complexes maintained higher levels of gene expression at day 8 and 10 compared to PEI/pDNA complexes (FIG. 7).

The accessibility of pDNA complexed with TAT was increased when CaCl₂ concentration was more than 350 mM (stock concentration). For PEI, CaCl₂ concentration >1000 mM seemed to increase pDNA accessibility to the dye (FIG. 8).

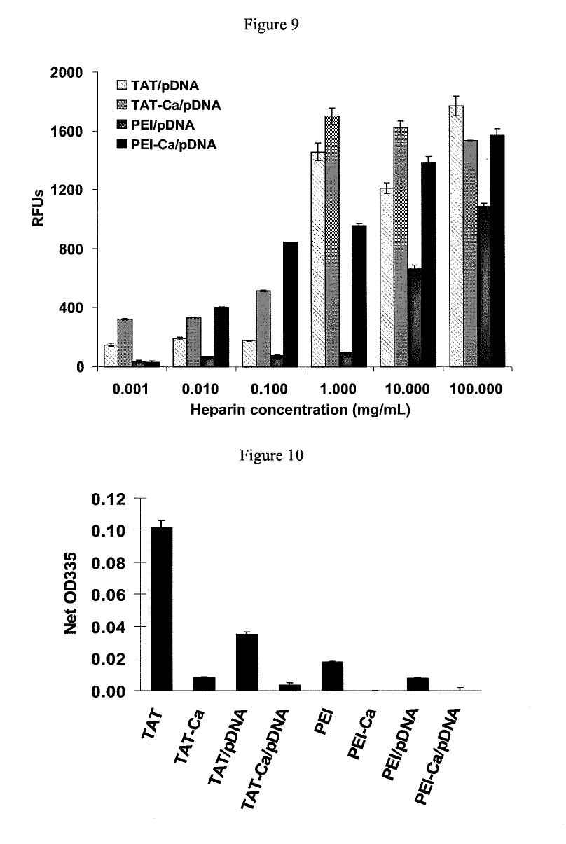

Unpackaging of the different complexes of TAT and PEI with and without CaCl₂ was determined by competitive binding using heparin. The complexes with CaCl₂ were more easily replaced by heparin (FIG. 9).

TNBS colorimetric assay was carried out to determine the degree of free amine groups of different pDNA complexes. The free amine groups in the complexes of TAT and PEI was appeared to be partially blocked by CaCl₂ (FIG. 10).

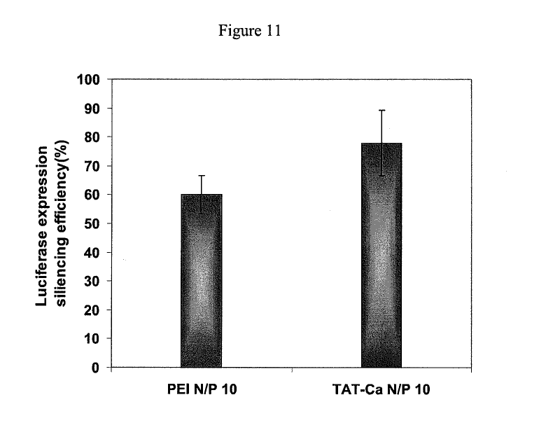

TAT-Ca complexes showed siRNA silencing of luciferase expression silencing (˜80% silencing) in A549 cells and were comparable to PEI complexes (FIG. 11).

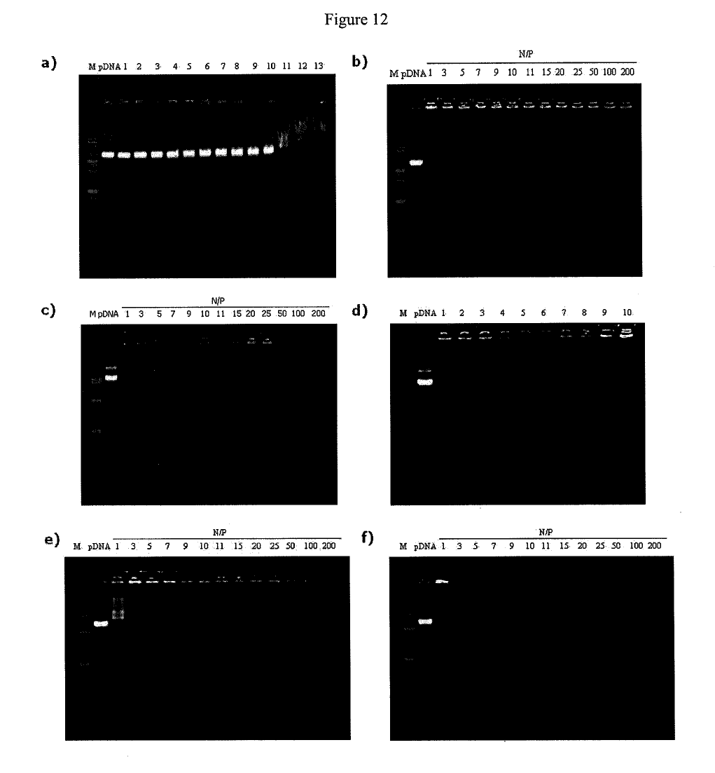

Gel electrophoresis results exhibited diminished fluorescence at lower as well as higher N/P ratios, indicating that that TAT and PEI completely covered and protected pDNA. In contrast, CaCl₂ showed no abilities to condense the plasmid DNA even at high concentration (1 M) (FIG. 12).

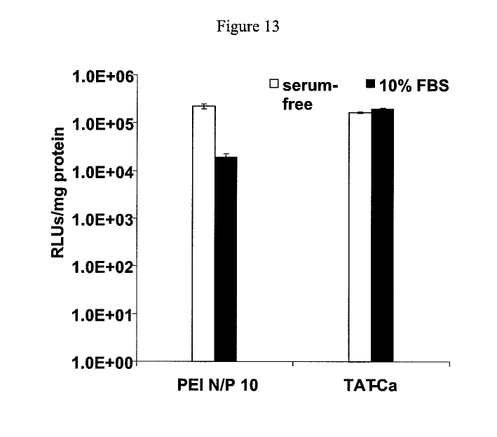

Serum did not significantly inhibit the transfection efficiency mediated by TAT-Ca complexes. In contrast, PEI complexes showed slightly decreased transfection efficiency in the presence of 10% FBS (FIG. 13).

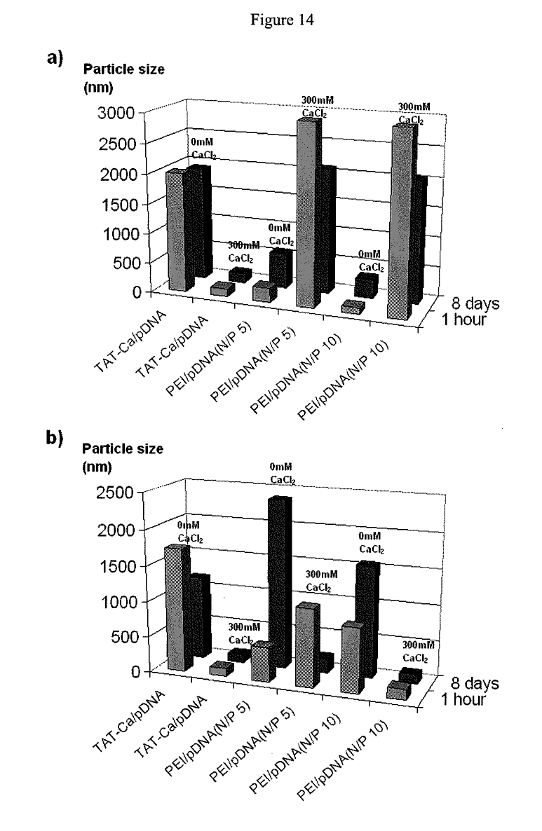

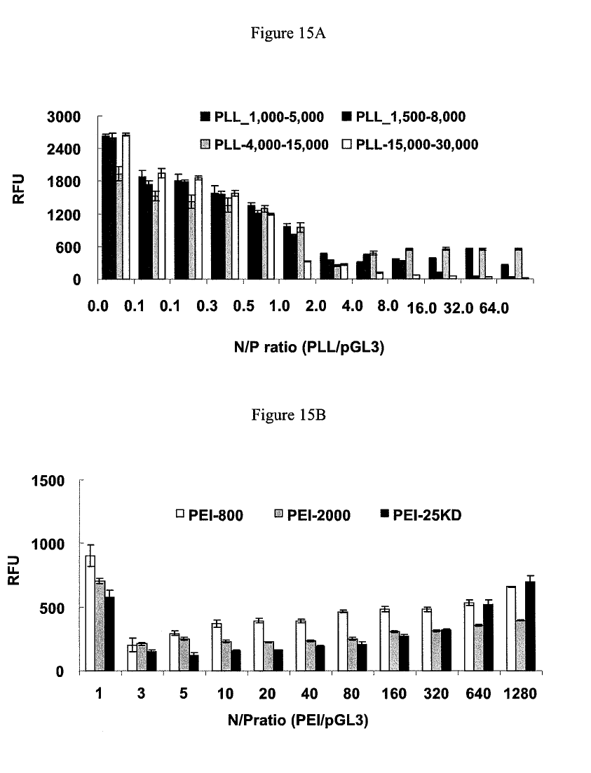

TAT/pDNA complexes without CaCl₂ caused particles to exhibit some agglomeration behavior in the absence (FIG. 14A) and presence of 10% FBS (FIG. 14B) over a period of 1 hour to 8 days. However, TAT-Ca/pDNA complexes showed good stability in serum-free and 10% FBS culture media during the same time frame. On the other hand, PEI/pDNA complexes remained stable in the absence of serum and CaCl₂ over a period of 8 days and retained their size (FIG. 14A), whereas the particle size of PEI-Ca/pDNA showed a decrease during the same time frame in the presence of 10% FBS (FIG. 14B).

### Example 2

Materials. SYBR Green was obtained from Invitrogen (Carlsbad, USA). Poly-L-lysine (PLL) hydrobromide, molecule weights 1,000-5,000, 1,500-8,000, 4,000-15,000, 15,000-30,000, Poly-L-arginine (PLA) hydrochloride, molecule weight 5,000-15,000, Poly-L-histidine (PLH) hydrochloride, molecule weight ≧5000, Protamine from salmon, Histone from calf thymus, PEI 25KD branch, PEI 800 K and PEI 2000 K, manganese sulfate monohydrate, and zinc chloride were purchased from Sigma-Aldrich (Saint Louis, USA). Luciferase assay kit was purchased from Promega (Madison, USA). TAT peptide was prepared by solid phase peptide synthesis in the lab. Calcium chloride, Nickel Chloride Hexahydrate, Nickel Sulfate Hexahydrate, MnCl₂, MnSO₄, MgCl₂ and MgSO₄, Cobalt (II) chloride, Cupric (II) chloride, Ferous (II) chloride were obtained from Fisher. Unless otherwise stated, water means ultrapure MilliQ water (resistance>18 MΩcm). Coomassie Plus™ Protein Assay kit was obtained from Pierce Biotechnology, IL.

Plasmid. Plasmid DNA encoding firefly luciferase enzyme (pGL3, 4.8 kbp) was obtained from Promega (Madison, Wis., USA). Plasmid cDNAs (pcDNA) were amplified in E. coli (DH5α) and purified using a plasmid Giga Kit (5) (Qiagen), and the concentration was determined photometrically at 260 nm.

Cell Cultures. Human lung carcinoma cell line A549 was purchased from American Type Culture Cell (Manassas, Va.). It was cultured in F-12K Medium (Kaighn's Modification of Ham's F-12 Medium), supplemented with 10% fetal bovine serum (FBS) and 1% (v/v) penicillin/streptomycin. The cells were cultured as monolayers in a humidified atmosphere of 95% air and 5% CO₂.

### Preparation of TAT/pDNA and Polyamine/pDNA Complexes. Briefly, 15 μl or 22.5 μl of TAT or polyamine in water was added into 10 μl or 15 μl or 10.1 mg/ml of pDNA in water and mixed by pipetting up and down. Then, 15 μl or 22.5 μl water or metal in water was added and mixed by pipetting up and down. Complexes were briefly spun down in a microcentrifuge. The complexes were allowed to form for 30 minutes at room temperature prior to use.

SYBR Green Assay for Polyamine/pDNA Complexes. The degree of pDNA accessibility following complexation with polyamine was assessed by the double-stranded-DNA-binding reagent SYBR Green (Invitrogen). Briefly, 10 μL (0.1 mg/mL) of pDNA was mixed with 15 μL of TAT or PEI solution, then 15 μl deionized water or metal solution was added. The complexes were allowed to form for 30 minutes at room temperature prior to use. After incubation, 120 μl deionized water and 160 μl 10× SYBR Green solution were added. Then, 80 μl of each sample was added to triplicate wells of 96-well cell culture plate. The plate was measured by a fluorescence plate reader (SpectraMax M5; Ex., 250 nm; Em., 520 nm).

TNBS Assay for Polyamine/pDNA Complexes. The degree of free amine group of polyamine accessibility following complexation with pDNA was measured by a colorimetric assay with 2,4,6-trinitro-benzenesulphonic acid (TNBS) as an assay reagent (Pierce). Briefly, 10 μl microliters of complex solution was added to 190 μl deionized water and then 200 μl of 0.02% TNBS solution in 0.1 M sodium bicarbonate buffer (pH 8.5) was added. The solution was rapidly mixed. After incubation at 37° C. for 2 hours, 80 μl of sample was added to triplicate wells of 96-well cell culture plate. The absorption at 335 nm was determined on a plate reader (SpectraMax M5).

In Vitro Cell Transfection Studies. A549 cells were plated on 96-well plates with approximately 8,000 cells/well and incubated in a humid 5% CO₂ incubator at 37° C. After 18-24 hours incubation, the medium was removed and washed with serum free cell culture medium one time. The cells were then treated with 100 μl sample (240 μl of serum free cell culture medium was added into 60 μl of complex for three wells). After the transfection for 5 hours, cells were further cultured with 100 μl of serum medium. After the indicated time, cells were washed once with PBS and lysed using 40 μl of lysis buffer per well. 20 μl of cell lysate was used to measure luciferase activity by the luciferase assay kit from Promega. 50 μl of luciferase assay reagent was added to measure light emission by plate reader (SpectraMax M5). Total cell protein concentration was determined by Coomassie Plus™ Protein Assay kit (Pierce Biotechnology, IL) with another 20 μl of cell lysate. Luciferase activity in each well was normalized to the relative light units (RLU) per mg of cell lysate proteins.

Cytoxicity Assay. Cytotoxicity of the complexes was determined by the CellTiter 96® Aqueous Cell Proliferation Assay kit (MTS assay) from Promega. A549 cells were plated on 96-well plates with approximately 8,000 cells/well and incubated in a humid 5% CO₂ incubator at 37° C. After 18-24 hours incubation, the medium was removed and the cells were washed with 100 μl serum free medium. Cells were treated with the samples for 12-24 hours. The serum free medium were then removed and replaced with a mixture of 100 μl fresh culture medium and 20 μl MTS reagent solution. The cells were incubated for 1-4 hours at 37° C. in 5% CO₂ incubator. Cell viability was assessed by measuring the absorbance at 490 nm by plate reader (SpectraMax M5) and expressed as the ratio of the A490 of cells treated with inhibitors over the control samples.

Transfection efficiency. Transfection efficiency of different CPPs and PEI complexes in A549 cells after 2 days {Antennapedia Heptapeptide (AHp), Antennapedia Leader peptide (ALp)} was studied using the peptides shown in Table 2.

TABLE 2

| CPP | Sequence |
|---|---|
| TAT | H-RKKRRQRRR-NH2 (SEQ ID NO: 5) |
| Arg7 | H-RRRRRRR-NH2 (SEQ ID NO: 6) |
| (polyarginine) | |
| Arg9 | H-RRRRRRRRR-NH2 (SEQ ID NO: 7) |
| Ahp (Antennapedia | H-RRMKWKK-NH2 (SEQ ID NO: 8) |
| Heptapeptide) | |
| Alp (Antennapedia | H-KKWKMRRNQFWVKVQRG-OH |
| Leader peptide) | (SEQ ID NO:9) |

Results

Condensation of pDNA by polyamine. The condensation of the pDNA (pGL3) by different molecular weights of PLL and PEI is shown in FIGS. 15A and B. The optimal N/P ratio of PLL and PEI were 2-4 and 2.5-10, respectively.

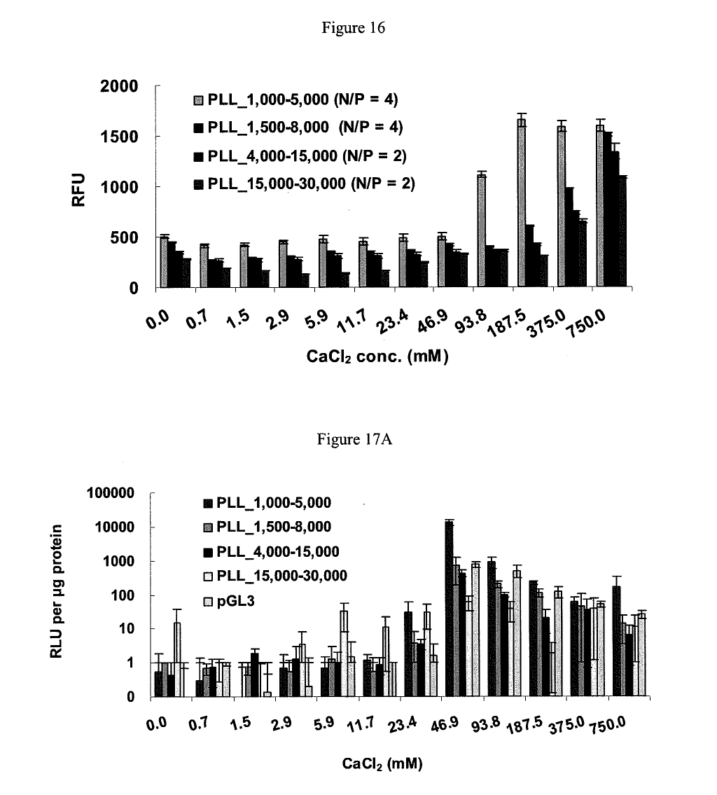

Effect of CaCl₂ concentration on the condensation of pDNA by polyamine. The condensation of the pDNA (pGL3) with PLL-1,000-5,000 was reduced when CaCl₂ concentration was more than 93.8 mM. For another three PLLs, the CaCl₂ concentration was started from 187.5 mM to reduce the condensation of the pDNA (pGL3) (FIG. 16).

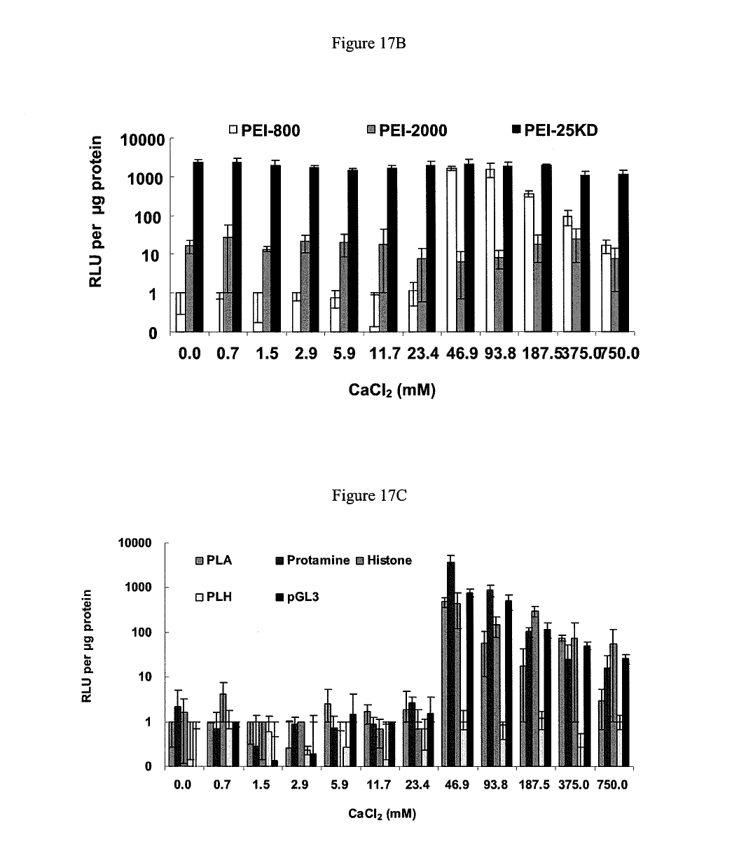

Effect of CaCl₂ concentration on the gene expression of pDNA/polyamine. Four types of PLL complexes showed highest gene expression around 46.9 mM CaCl₂ in the complex (stock conc. of CaCl₂ is 125 mM) (FIG. 17A). PLA, protamine, histone and PEI-800 showed similar results (FIG. 17B and FIG. 17C). However, gene expressions of PEI-2,000 and PEI 25KD were not affected by CaCl₂ concentration (FIG. 17B).

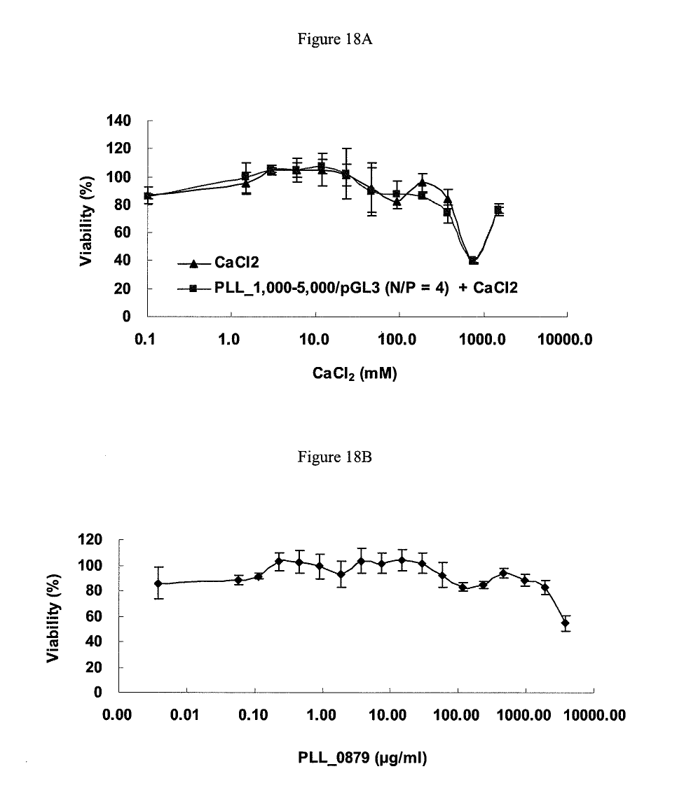

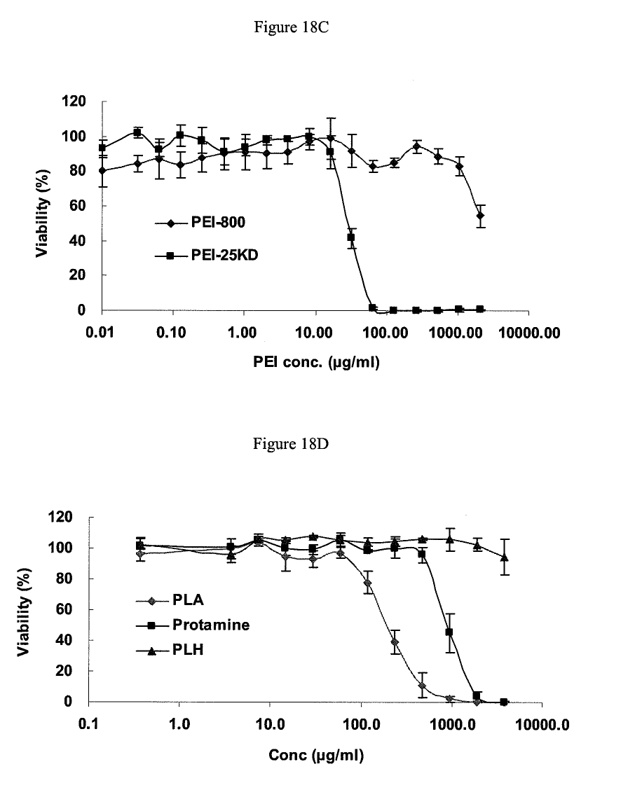

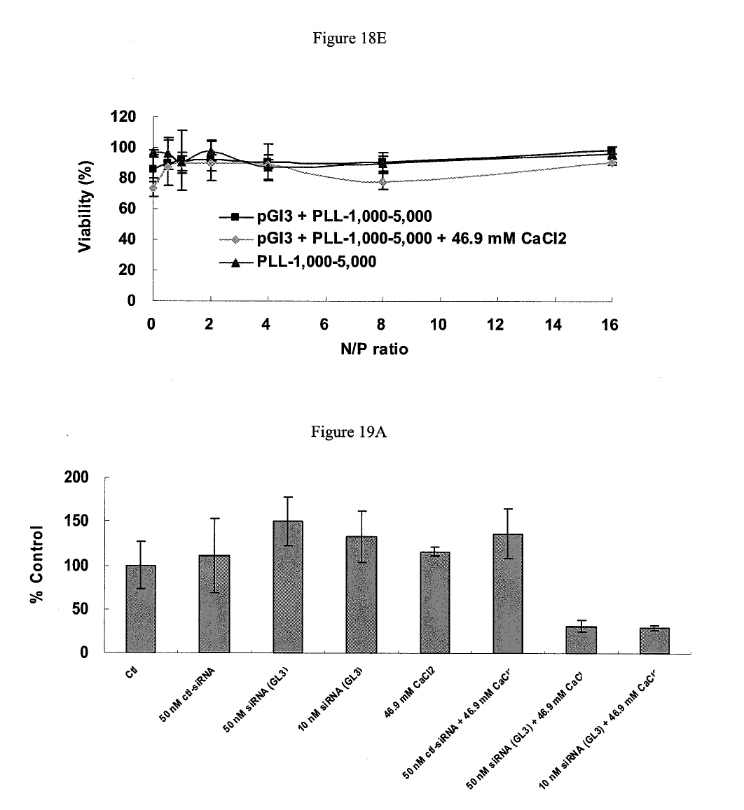

Cytotoxicity Assay. PEI-25KD, PLA and protamine showed cytotoxicity on the A549 cells (FIGS. 18A and 18B). IC50 was 30, 201, and 890 μg/ml, respectively. PLH did not show cytotoxicity at the test conditions (FIG. 18B). The IC50s of PEI-800 and PLL-1,000-5,000 were higher than the highest concentrations tested (FIGS. 18A and 18C). PLL-1,000-5,000 and its complexes with CaCl₂ and without CaCl₂ at the concentration of 400 μg/ml did not show any cytotoxicity (FIG. 18D). 46.9 mM CaCl₂ only or in the complex did not show cytotoxicity on A549 cells too (FIG. 18D and FIG. 18E).

Transfection efficiency of different types of cell penetrating peptides in the presence of CaCl₂ (CPPs-Ca) in A549 cells after 2 days incubation showed higher level of gene expression (FIG. 20).

### Example 3

Materials. Poly-L-lysine (PLL) hydrobromide, molecule weight 1,000-5,000, protamine from salmon and branched PEI 25KD were purchased from Sigma-Aldrich (Saint Louis, USA). Luciferase assay kit was purchased from Promega (Madison, USA). TAT peptide was prepared by solid phase peptide synthesis in the lab. Calcium chloride was obtained from Fisher. Unless otherwise stated, water means ultrapure MilliQ water (resistance>18 MΩ cm). Coomassie Plus™ Protein Assay kit was obtained from Pierce Biotechnology, IL. The 21-nucleotide long luciferase siRNA GL3 and negative control siRNA were purchased from Ambion. The firefly luciferase gene of the pGL3-basic plasmid, the Renilla luciferase plasmid pGL4.75 and dual luciferase reporter assay system were from Promega.

Preparation of PEI/DNA (N/P=10) Complexes. Briefly, 15 μl 0.1 mg/ml pDNA containing a pGL3 and pGL4.75 mixture (ratio of pGL3 with pGL4.75 is 39/1) in water was added into 22.5 μl PEI in water and mixed by pipetting up and down. And then 22.5 μl water was added and mixed by pipetting up and down. Complexes were briefly spun down in a microcentrifuge and incubated for 30 min at room temperature prior to use.

Preparation of Polyamine/siRNA Complexes. Briefly, 10 μl siRNA in water was added into 15 μl peptide TAT or other polyamine in water and mixed by pipetting up and down. And then 35 μl water or CaCl₂ solution was added and mixed by pipetting up and down. Complexes were briefly spun down in a microcentrifuge and incubated for 30 minutes at room temperature prior to use. The N/P ratio of peptide TAT, PEP and PLL_{—}1,000-5,000 with siRNA were 30, 10 and 5, respectively. The concentration of protamine for condensation of siRNA was 7.5 μg/ml. The final concentration of CaCl₂ in the complex was 46.9 mM. The siRNA concentrations in the complex were 50 and 250 nM.

Preparation of Polyamine/pGL3, pGL4.75 and siRNA Complexes for Cotransfection. Briefly, 15 μl pGL3, pGL4.75 and siRNA mixture (ratio of pGL3 with pGL4.75 was 4/1) in water was added into 22.5 μl peptide TAT or polyamine in water and mixed by pipetting up and down. And then 22.5 μl water or CaCl₂ solution was added and mixed by pipetting up and down. Complexes were briefly spun down in a microcentrifuge and incubated for 30 min at room temperature prior to use. The N/P ratio of peptide TAT, PEP and PLL 1,000-5,000 with siRNA were 30, 10 and 4, respectively. The concentration of protamine for condensation of siRNA was 7.5 μg/ml. The final concentration of CaCl₂ in the complex was 46.9 mM. The siRNA concentrations in the complex were 25, 50, 125 and 250 nM.

Preparation of Polyamine/pGL3 and siRNA Complexes for Cotransfection. Briefly, 15 μl pGL3 and siRNA mixture in water was added into 22.5 μl peptide TAT or polyamine in water and mixed by pipetting up and down. And then 22.5 μl water or CaCl₂ solution was added and mixed by pipetting up and down. Complexes were briefly spun down in a microcentrifuge and incubated for 30 mM at room temperature prior to use. The N/P ratio of peptide TAT, PEP and PLL_{—}1,000-5,000 with pGL3 and siRNA were 30, 10 and 4, respectively. The final concentration of CaCl₂ in the complex was 46.9 mM. The siRNA concentrations in the complex were 25, 50, 125 and 250 nM.

Individual Transfection of pDNA and siRNA Complexes. A549 cells were plated on 96-well plates with approximately 8,000 cells/well and incubated in a humid 5% CO₂ incubator at 37° C. After 18-24 hours incubation, the medium was removed and washed with serum free cell culture medium one time. The cells were then treated with 100 μl sample (240 μl of serum free cell culture medium was added into 60 μl of PEI/DNA (N/P=10) complex for three wells). After the transfection for 4 hours, cells were further cultured with 100 μl of serum medium for 20 hours. The medium was removed and washed with serum free cell culture medium again. The cells were then treated with siRNA complex (240 μl of serum free cell culture medium was added into 60 μl of polyamine/siRNA complex for three wells) for 5 hours. After the indicated time, cells were washed once with PBS and lysed using 40 μl of passive lysis buffer per well. 20 μl of cell lysate was used to measure luciferase activity by the dual luciferase reporter assay system (Promega). 50 μl of LAR II reagent was added to measure light emission of firefly luciferase by plate reader (SpectraMax M5). Another 50 μl of Stop & GLO reagent was added to measure light emission of Renilla luciferase by plate reader. Total cell protein concentration was determined by Coomassie Plus™ Protein Assay kit (Pierce Biotechnology, IL) with another 20 μl of cell lysate. Luciferase activity in each well was normalized to the relative light units (RLU) per μg of cell lysate proteins.

Co-Transfection of pGL3, pGL4.75 and siRNA Complexes. A549 cells were plated on 96-well plates with approximately 8,000 cells/well and incubated in a humid 5% CO₂ incubator at 37° C. After 18-24 hours incubation, the medium was removed and washed with serum free cell culture medium one time. The cells were then treated with 100 μl sample (240 μl of serum free cell culture medium was added into 60 μl of complex for three wells). After the transfection for 5 hours, cells were further cultured with 100 μl of serum medium. After the indicated time, cells were washed once with PBS and lysed using 40 μl of lysis buffer per well. After the indicated time, cells were washed once with PBS and lysed using 40 μl of passive lysis buffer per well. Luciferase activity of firefly luciferase and Renilla luciferase were measured by above method.

Co-Transfection of pGL3 and siRNA Complexes. A549 cells were plated on 96-well plates with approximately 8,000 cells/well and incubated in a humid 5% CO₂ incubator at 37° C. After 18-24 hours incubation, the medium was removed and washed with serum free cell culture medium one time. The cells were then treated with 100 μl sample (240 μl of serum free cell culture medium was added into 60 μl of complex for three wells). After the transfection for 5 h, cells were further cultured with 100 μl of serum medium. After the indicated time, cells were washed once with PBS and lysed using 40 μl of lysis buffer per well. After the indicated time, cells were washed once with PBS and lysed using 40 μl of passive lysis buffer per well. Luciferase activity of firefly luciferase was measured by above method.

Results

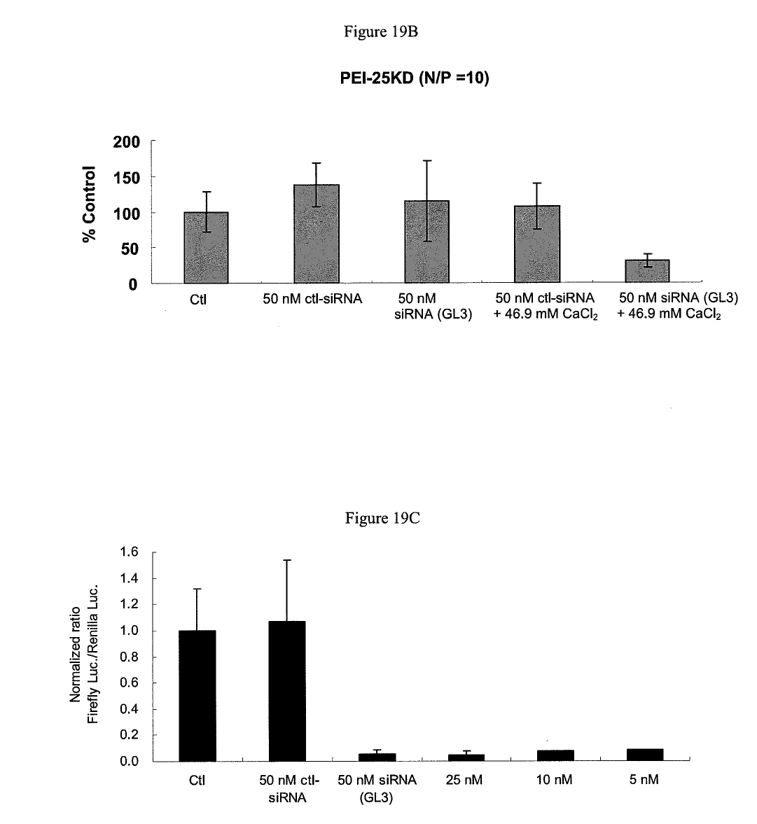

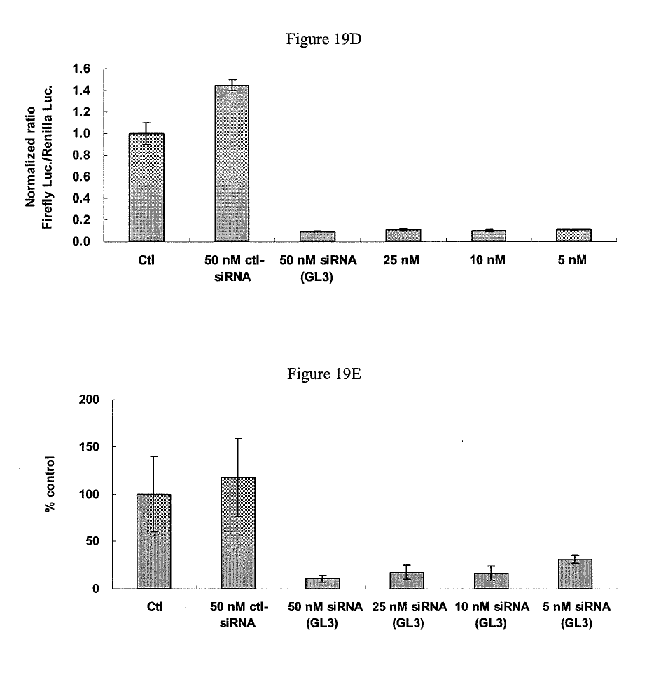

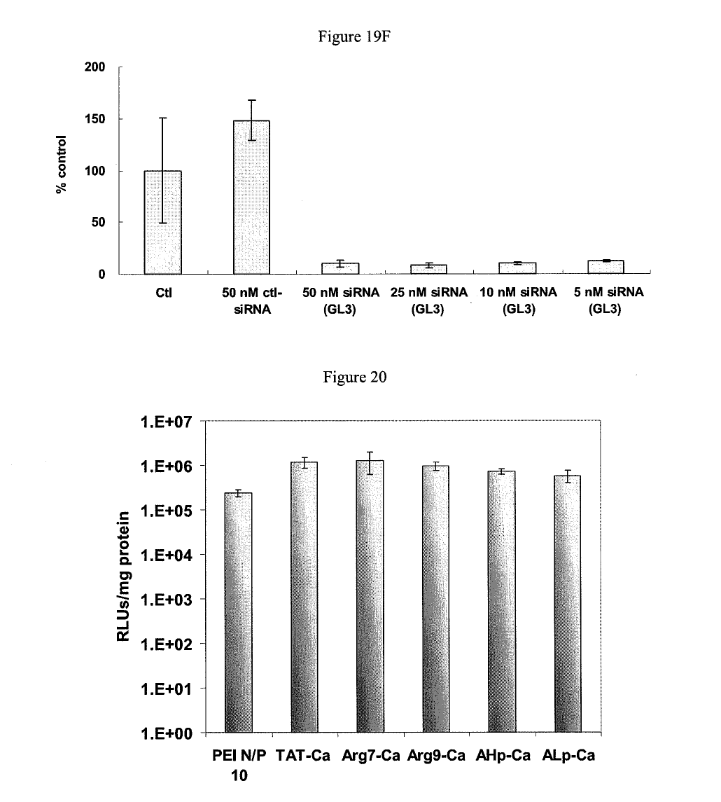

Firefly and Renilla Luciferase activities were measured 1-3 days after transfection siRNA complexes of TAT or PEI (FIGS. 19A-C). Control siRNA (nonspecific siRNA) as the negative control did not inhibit Firefly luciferase activity. In the presence of CaCl₂, the siRNA (GL3) showed specific inhibition of Firefly luciferase gene expression, inhibited by 75% with 50 nM siRNA (GL3) compared with Renilla luciferase (internal control). Expression of Renilla luciferase was unaffected by the presence of siRNA, suggesting that inhibition is specific to the target gene. Without CaCl₂ the siRNA (GL3) did not show specific inhibition of Firefly luciferase gene expression (FIGS. 19A-C).

Similar results were obtained by cotransfection of siRNA, pGL3, and pGL4.75 complexes with TAT or PEI in the presence of CaCl₂. Inhibitory effects are more than 90% by 50 nM siRNA (GL3) both with TAT and PEI complexes (FIGS. 19D and 19E).

Cotransfection of siRNA and pGL3 complexes by TAT and PEI in the presence of CaCl₂ were carried out. More than 90% inhibitory effects were obtained with 50 nM siRNA (GL3) (FIGS. 19F and 19G).

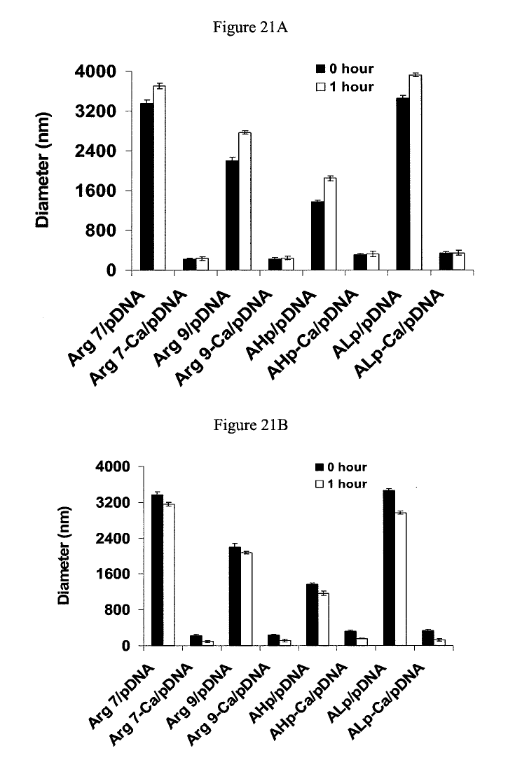

CPPs/pDNA complexes without CaCl₂ caused particles to exhibit some agglomeration in the absence and presence of 10% FBS over a period of Oh to 1 h. However, CPPs-Ca/pDNA complexes exhibited good stability in serum-free and serum-containing culture media during the same time frame (FIGS. 21A and 21B).

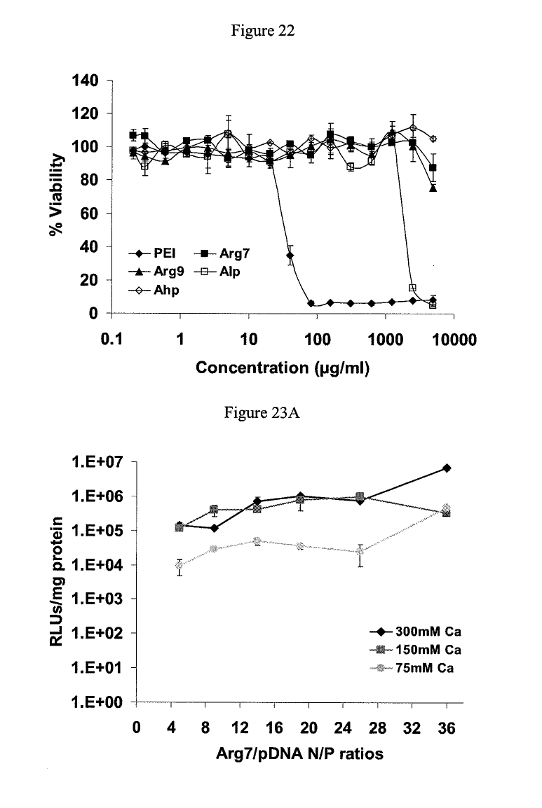

CPPs peptides revealed minimal evidence of cytotoxic effects. Alp exhibited very little cytotoxicity at high concentration (IC₅₀˜2144 μg/mL) and cells maintained a high viability, while branched PEI polymer induced a great deal of cell death (IC₅₀˜35 μg/mL) (FIG. 22).

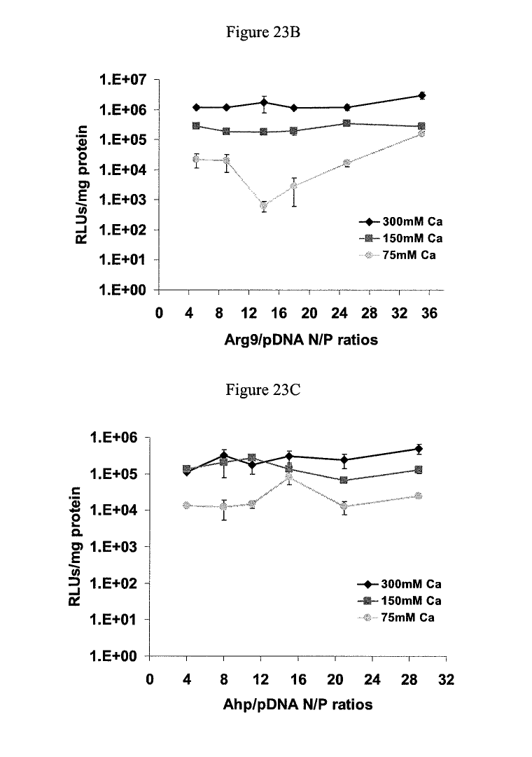

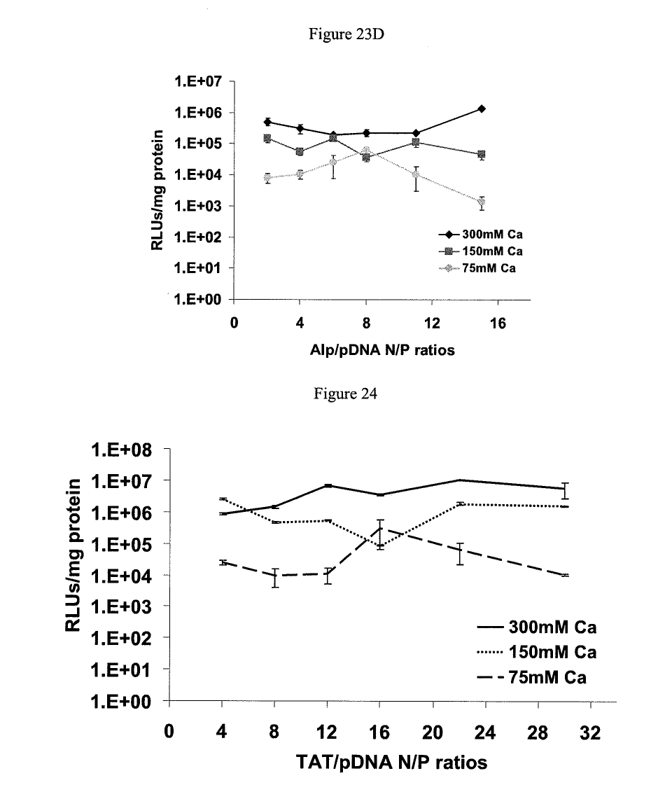

Luciferase gene expression complexed with CPPs was evaluated 1 day after transfection as a function of the concentration of CaCl₂ and N/P ratios. CPPs complexes showed a higher level of gene expression at 300 mM of added CaCl₂ (final concentration ˜115 mM) compared with those complexes at 75 and 150 mM CaCl₂ (FIGS. 23a, 23b, 23c, and 23d).

Luciferase gene expression complexed with TAT was evaluated 1 day after transfection as a function of the concentration of CaCl₂ and N/P ratios. TAT complexes showed a higher level of gene expression at 300 mM of added CaCl₂ compared with those complexes at 75 and 150 mM CaCl₂ (FIG. 24).

Luciferase gene expression complexed with TAT₂, TAT₃, TAT₄, and TAT₅ was evaluated 1 day after transfection as a function of the concentration of CaCl₂ and N/P ratios. The sequences of TAT₂, TAT₃, TAT₄, and TAT₅ are shown below in Table 3.

TABLE 3

| Peptide | Sequence |
|---|---|
| TAT 2 | H-RKKRRHRRR-NH2 (SEQ ID NO: 10) |
| TAT 3 | H-RKKRRQHRRR-NH2 (SEQ ID NO: 11) |
| TAT 4 | H-RRKRRQHRRR-NH2 (SEQ ID NO: 12) |
| TAT 5 | H-RKKRRQRRRHRRKKR-NH2 (SEQ ID NO: 13) |

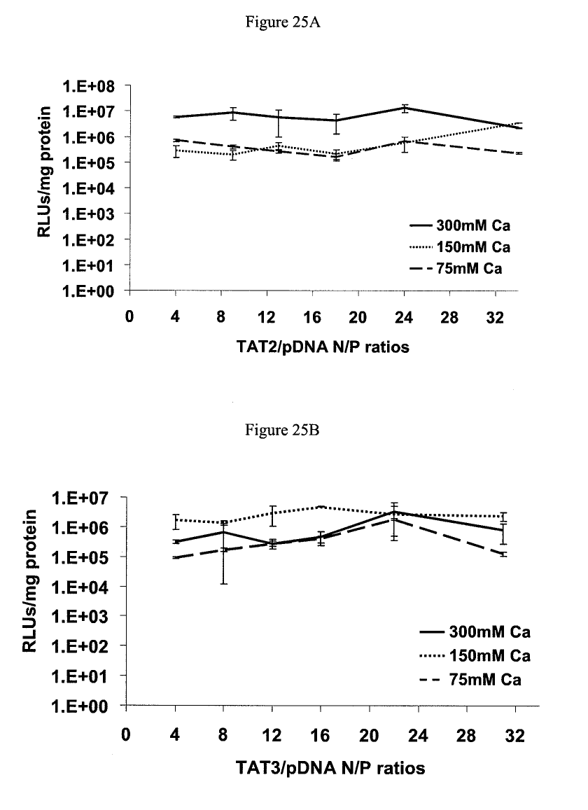

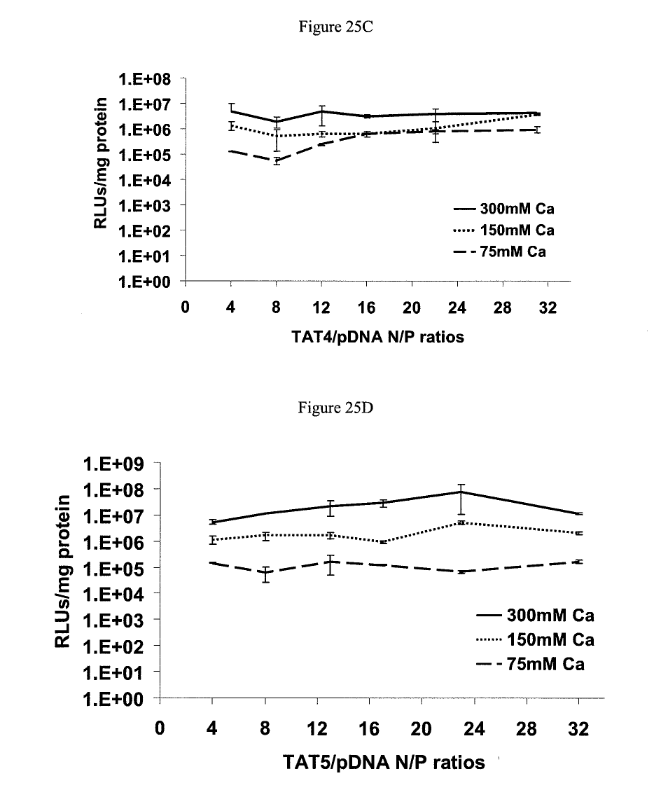

TAT₂, TAT_{S}, and TAT₄ complexes showed a higher level of gene expression at 150 mM CaCl₂, however TAT₅ complexes revealed a higher level of gene expression at 300 mM CaCl₂ (FIGS. 25A-25D).

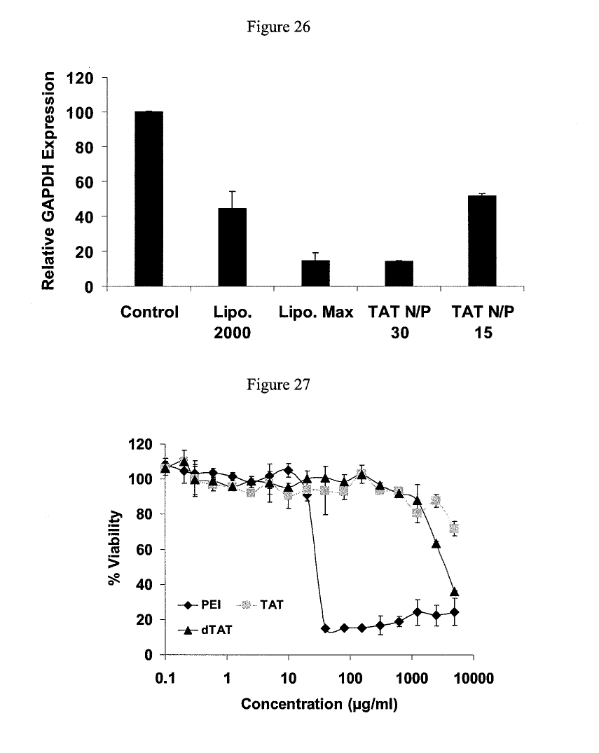

TAT-Ca complexes showed successful delivery of siRNA (GAPDH) into HeLa cells with high silencing efficiency (˜80%) compared to Lipofectamine 2000, and Lipofectamine RNAiMAX complexes (FIG. 26).

### Example 4

Materials. Anti-luciferase siRNA-1 (MW 13358 g/mol) and siRNA control (non-targeting) were supplied by Thermo Scientific Dharmacon® (Chicago, Ill.). TAT peptide (RKKRRQRRR (SEQ ID NO: 14; Mw=1338.6 Da) and dTAT (RKKRRQRRRHRRKKR SEQ ID NO 15; Mw=2201.7 Da) peptide were purchased from Biomatik Corporation (Cambridge, Ontario, Canada). Branched polyethylenimine (PEI, 25 kDa) was obtained from Aldrich (Milwaukee, Wis.). Calcium chloride (CaCl₂.2H₂O), Nuclease-free water and BCA™ Protein Assay were purchased from Fisher Scientific (Pittsburgh, Pa.). Glucose was acquired from Sigma. A549-luc-C8 Bioware® cell line was obtained from Caliper LifeSciences (Hopkinton, Mass.). The cell culture medium (RPMI-1640) and (Ham's F-12 Nutrient Mixture, Kaighn's modified with L-glutamine) were purchased from the American Type Culture Collection (ATCC, Rockville, Md.). Heat inactivated fetal bovine serum (FBS) was purchased from Atlanta Biologicals (Lawrenceville, Ga.). Penicillin-streptomycin was purchased from MB Biomedical, LLC (Solon, Ohio). Trypsin-EDTA was purchased through Gibco (Carlsbad, Calif.). MTS reagent [tetrazolium compound; 3-(4,5-dimethylthiazol-2-yl)-5-(3-carboxymethoxyphenyl)-2-(4-sulfophenyl)-2H-tetrazolium, inner salt] and Luciferase Assay System were purchased from Promega (Madison, Wis.).

Preparation of Complexes. dTAT and PEI Complexes were Prepared essentially as described previously (Baoum et al., 2009; Baoum and Berkland). Briefly, various amounts of polycations and siRNA were first dissolved in known volume of nuclease-free water (NFW). Ten microliters (e.g., 10 nM) of siRNA solution was added rapidly to fifteen microliters polycation (TAT, dTAT or PEI) solution while pipetting. To this solution, fifteen microliters (e.g., 23.1 mM) CaCl₂ (or NFW in the case of PEI) was added and mixed by vigorous pipetting. This resulted in different N/P ratios of polycation/siRNA complexes. The complexes then were allowed to form during 20 minute incubation at 4° C. prior to use. Complexes were freshly prepared before each individual analysis.

Size and Zeta potential measurement. The effective hydrodynamic diameter of the complexes was analyzed using a dynamic light scattering (DLS) system (Brookhaven Instrument, Holtsville, N.Y.) equipped with a 50 mW HeNe laser operating at 532 nm. The complexes were prepared at a constant pDNA concentration of 100 μg/mL whereas the N/P ratios of the complexes were varied. The scattered light was monitored at 90° to the incident beam. For each sample, the data was collected continuously for three 1-minute intervals. The diameter of the complexes was obtained from the diffusion coefficient by the Stokes-Einstein equation using the method of cumulants. Zeta potential measurements were obtained by phase analysis light scattering using a Brookhaven Zeta PALS instrument. The electrophoretic mobility of the samples was determined from the average of 10 cycles of an applied electric field. The zeta potential was determined from the electrophoretic mobility from the Smoluchowski approximation.

Cell culture. Culturing of human epithelial lung cell line A549-luc-C8, stably expressing luciferase, was performed according to the protocol provided by Caliper LifeSciences. A549-luc-C8 cells were grown in RPMI-1640 supplemented with 10% v/v heat inactivated FBS and 1% v/v Penicillin/streptomycin at 37° C. in a humidified air atmosphere containing 5% CO₂.

In vitro luciferase gene knockdown studies. A549-luc-C8 cells were trypsinized, counted and diluted to a concentration of approximately 100,000 cells/mL. Then, 0.1 mL of that dilution was added to each well of a 96-well plate and the cells were incubated in a humidified atmosphere at 5% CO₂ and 37° C. Twenty-four hours before transfection, the cells were washed once with PBS and 100 μl of sample at 10, 25 or 50 nM siRNA concentration (20% of complex to 80% of serum-free cell culture medium (Ham's F-12 Nutrient Mixture, Kaighn's modified with L-glutamine)) was added to each well. Cells were incubated with the complexes for 5 hours. The media was then removed by aspiration and 100 μL of fresh serum medium (RPMI-1640) was added followed by further incubation (48 hours). In addition to the anti-luc siRNA, a non-silencing siRNA sequence was used to ensure that the decrease in luciferase expression was due to the anti-luc siRNA and not to cytotoxicity effects of the vector. The Luciferase Assay System from Promega was used to determine luciferase gene silencing following the manufacturer's recommended protocol. The light units were normalized against protein concentration in the cells extracts, which were measured using the BCA™ Protein Assay. The data were expressed as a percentage of control (non-specific siRNA control).

Assessment of cytotoxicity (MTS Assay). The cytotoxicity of polymers was determined by the CellTiter 96® Aqueous Cell Proliferation Assay (Promega). A549-luc-C8 cells were grown as described in the transfection experiments. Cells were treated with the TAT, dTAT or PEI for approximately 24 hours. The media were then removed and replaced with a mixture of 100 μL fresh culture media (RPMI-1640) and 20 μL MTS reagent solution. The cells were incubated for 3 hours at 37° C. in the 5% CO₂ incubator. The absorbance of each well was then measured at 490 nm using a microtiter plate reader (SpectraMax, M25, Molecular Devices Corp., CA) to determine cell viability.

Assessment of toxicity in vivo. Stock solutions of 0.3 M CaCl₂, 10% Glucose, and 174 mg/ml dTAT were prepared. Non-silencing control siRNA (Dharmacon; Sense: UGUACUGCUUACGAUUCGGtt; SEQ ID NO 16, Antisense: CCGAAUCGUAAGCAGUACAtt; SEQ ID NO 17) was dissolved in nuclease-free water (61.5 nM). The dTAT/siRNA complexes were prepared in 1.5 ml microcentrifuge tubes by mixing stock solutions (at concentrations indicated above). Formulation 1 used 15 μL of dTAT, 10 μL siRNA, 25 μL Glucose, and 5 μl CaCl₂. Each ingredient was added to the previous component(s) and mixed by pipetting between each addition. Four of these formulas were individually prepared and kept at 4° C. for 20 minutes, then kept at room temperature for 5 minutes prior to injection. Remaining formulations (2-10) were prepared by a serial 1:2 dilution of stock siRNA solution and stock dTAT solution following the same experimental protocol as described above. The dTAT/siRNA complexes (volume=260 μl) were prepared for each individual animal and each 0.2 mL injection volume contained approximately the following amounts of dTAT peptide (mg/ml): 40, 20, 10, 5, 2.5, 1.25, 0.64, 0.32, 0.16, 0. Each formulation was administered via 200 μL injection into the tail vein of 12 week old male Balb/C mice (10 animals total).

Assessment of knockdown and biodistribution. Tissue biodistribution and functional siRNA delivery efficiency of dTAT/siRNA complexes were tested in vivo. Glyceraldehyde 3-phosphate dehydrogenase (GAPDH) siRNA is a sequence that is not expressed in mice, which allows quantitative measurement of siRNA accumulation. Quantitation of siRNA-induced gene silencing in tissues was performed by administration of GAPDH siRNA and a scrambled sequence (negative control) siRNA. GAPDH mRNA expression levels were determined from selected tissues.

The dTAT/siRNA complexes were prepared similarly to the toxicity study except that a 94.6 mg/mL stock solution of the dTAT peptide and a 500 μM stock solution of siRNA were used. Nuclease free water was also added to dilute the 500 μM stock solution of siRNA to attain the lower siRNA dose. The final concentration of siRNA was 77 or 38.5 μM. Each formulation was administered via 200 μL injection into the tail vein of 8-12 week old male Balb/C mice (12 animals total; 3 per group). Forty eight hours after administration, animals were sacrificed and tissues extracted (brain, liver, kidney, lung, muscle, stomach).

Tissues were homogenized and processed using a miRvana isolation kit that allows isolation of both total protein fraction and total RNA fraction (qRT-PCR analysis) from the same tissue. A TaqMan qRT-PCR assay was performed on all isolated mouse samples (duplicate RT step followed by a single PCR step). The geometric mean of two genes, 18S and CyclophilinA, were used to normalize the raw GAPDH mRNA data. A qRT-PCR method using sequence-specific primers was also employed to quantify GAPDH siRNA delivered to these tissue samples. The amount of GAPDH siRNA was normalized to the appropriate miR-24 value and then calculated as percent accumulation relative to the tissue samples from animals given PBS.

Statistical analysis. Statistical evaluation of data was performed using an analysis of variance (one-way ANOVA). Newman-Keuls was used as a post-hoc test to assess the significance of differences. To compare the significance of the difference between the means of two groups, a t-test was performed; in all cases, a value of p<0.05 was accepted as significant.

Results

Particle Sizes and Zeta potentials of TAT, dTAT and PEI complexes. TAT, dTAT and PEI complexes were prepared by mixing siRNA with each polycation at various N/P ratios. These complexes were thoroughly mixed by pipetting and CaCl₂ was added (final concentration 23.1-69.2 mM). The size of the complexes prepared with 25 nM of siRNA was determined by DLS (Table 4A and 4B). In general, the added CaCl₂ produced small TAT and dTAT complexes at all N/P ratios (58.5-201.3 nm) with polydispersity values below 0.24. The size of the TAT and dTAT complexes generally decreased with increasing N/P ratios and increasing concentrations of calcium. The zeta potential of TAT and dTAT complexes was ˜15 mV. The charge did not substantially change with the N/P ratio. In comparison, PEI complexes showed a small particle size (90 nm) with a higher zeta potential of ˜20 mV. Calcium was not used to prepare the PEI complexes.

TABLE 4A

| TAT-N/P (Ca mM) | Diameter (nm) | Polydispersity |
|---|---|---|
| 7 (0.00) | 1012.0 ± 23.4 | 0.265 |
| 7 (23.1) | 139.7 ± 3.9 | 0.108 |
| 7 (34.6) | 113.7 ± 3.7 | 0.211 |
| 7 (69.2) | 116.6 ± 11.3 | 0.125 |
| 18 (0.00) | 1411.3 ± 54.1 | 0.195 |
| 18 (23.1) | 112.0 ± 6.9 | 0.230 |
| 18 (34.6) | 99.9 ± 10.8 | 0.134 |
| 18 (69.2) | 60.6 ± 7.5 | 0.139 |
| 25 (0.00) | 2105.0 ± 87.9 | 0.263 |
| 25 (23.1) | 144.2 ± 2.1 | 0.222 |
| 25 (34.6) | 117.8 ± 9.7 | 0.178 |
| 25 (69.2) | 59.3 ± 6.4 | 0.203 |
| 33 (0.00) | 2210.0 ± 76.9 | 0.271 |
| 33 (23.1) | 152.9 ± 8.1 | 0.169 |
| 33 (34.6) | 122.8 ± 4.5 | 0.225 |
| 33 (69.2) | 58.5 ± 7.3 | 0.189 |

TABLE 4B

| dTAT-N/P (Ca mM) | Diameter (nm) | Polydispersity |
|---|---|---|
| 6 (0.00) | 1170.4 ± 90.4 | 0.199 |
| 6 (23.1) | 124.3 ± 9.4 | 0.143 |
| 6 (34.6) | 101.7 ± 15.2 | 0.179 |
| 6 (69.2) | 98.5 ± 6.8 | 0.201 |
| 17 (0.00) | 2256.5 ± 45.1 | 0.253 |
| 17 (23.1) | 189.3 ± 5.1 | 0.230 |
| 17 (34.6) | 203.4 ± 9.4 | 0.177 |
| 17 (69.2) | 152.0 ± 13.6 | 0.215 |
| 23 (0.00) | 1978.2 ± 68.2 | 0.223 |
| 23 (23.1) | 155.7 ± 4.2 | 0.242 |
| 23 (34.6) | 113.4 ± 7.9 | 0.196 |
| 23 (69.2) | 89.7 ± 5.1 | 0.103 |
| 31 (0.00) | 2190.5 ± 39.6 | 0.247 |
| 31 (23.1) | 197.4 ± 8.3 | 0.122 |
| 31 (34.6) | 145.2 ± 1.9 | 0.108 |
| 31 (69.2) | 90.1 ± 3.3 | 0.127 |

Cytotoxicity of TAT, dTAT and PEI complexes. Efficient delivery together with low cytotoxicity is extremely desirable to translate RNAi therapeutic vectors. To evaluate the cytotoxicity of free TAT, dTAT, and PEI, an MTS assay was performed by incubating A549-luc-C8 cells with up to 5 mg/mL of TAT, dTAT or PEI for 24 hours (FIG. 27). TAT peptide revealed almost no evidence of cytotoxicity and cells maintained high viability, while dTAT showed modest cytotoxicity (IC₅₀˜4000 μg/mL). The branched PEI induced substantial cytotoxicity (IC₅₀ of 22 μg/mL) as expected.

In vitro luciferase gene knockdown by TAT, dTAT and PEI complexes. The silencing efficiency of TAT and dTAT complexes was investigated using the human lung carcinoma cell line A549-luc-C8. This cell line stably expresses firefly luciferase. Luciferase knockdown was evaluated 48 hours after treatment with the TAT, dTAT or PEI polyplexes. The data were normalized to the luciferase protein levels of cells treated with control siRNA complexes. Complexes prepared at five different N/P ratios of TAT/siRNA or dTAT/siRNA were condensed by adding different concentrations of CaCl₂ (23.1, 34.6, or 69.2 mM) after complex formation. Different siRNA doses (10, 25 or 50 nM) were studied and compared to PEI polyplexes (N/P 10).

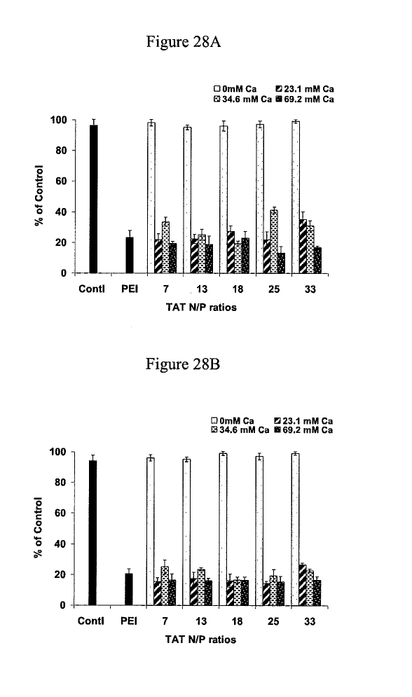

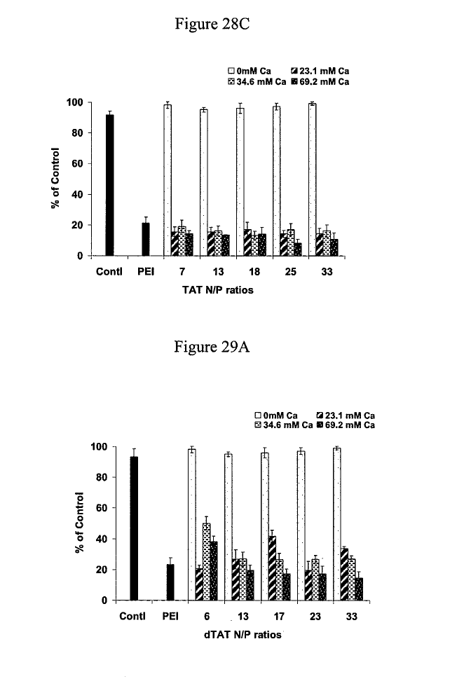

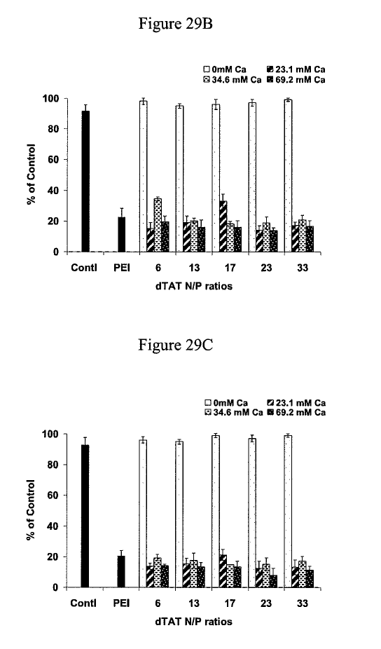

In general, TAT and dTAT complexes showed a slightly higher level of luciferase knockdown for the various N/P ratios and CaCl₂ concentrations when compared to PEI, which showed excellent knockdown in the absence of CaCl₂ (FIGS. 28 and 29). The level of luciferase knockdown of TAT and dTAT complexes seemed to depend on N/P ratio and CaCl₂ concentration. TAT and dTAT typically showed the greatest gene silencing at high calcium concentration (69.2 mM) and moderately high N/P ratios (N/P ratios of 25 and 23, respectively). The luciferase knockdown of PEI complexes was found to be somewhat independent of the siRNA dose. Strikingly, no luciferase knockdown was observed for TAT and dTAT complexes without CaCl₂. It is important to note that the TAT and dTAT siRNA control complexes including 69.2 mM CaCl₂ did not affect luciferase expression levels, which further supported the premise that these vectors did not influence the viability of A549-luc-C8 cells.

In vivo dose escalating toxicity study. The CPP dTAT was selected for further studies in vivo. A dose escalating study was conducted in an effort to establish a lethal dose (LD₅₀) for the dTAT peptide. For these studies, only a minimal amount of siRNA was included so that animal responses would be indicative of the toxicity of dTAT. Animals were administered the formulations via tail vein injection (200 μL) at doses up to 1,000 mg/kg of dTAT (Table 5). The highest dose yielded a transient behavioral response, with the animal fully recovering.

TABLE 5

| | | | Final siRNA | |
|---|---|---|---|---|
| | | Total | concentration | Toxicity/ |
| Animal | dTAT | dose | (200 μL injection | Behavioral |
| ID | (mg/mL) | (mg/Kg) | volume) (nM) | Effects |
| 1 | 40 | 1000 | 9.46 | *Behavioral |
| | | | | Phenotype |
| 2 | 20 | 500 | 4.73 | Normal |
| 3 | 10 | 250 | 2.36 | Normal |
| 4 | 5 | 125 | 1.18 | Normal |
| 5 | 2.5 | 62.5 | 0.59 | Normal |
| 6 | 1.25 | 31.25 | 0.29 | Normal |
| 7 | 0.64 | 15.63 | 0.14 | Normal |
| 8 | 0.32 | 7.81 | 0.07 | Normal |
| 9 | 0.16 | 3.90 | 0.03 | Normal |
| 10 | 0.00 | 0.00 | 0.00 | Normal |

*Behavioral Phenotype: Mouse was less active after injection, laid down for 1 hour, dragged back legs. During 8 hours post-injection animal was less alert and moving compared to other animals. Resumed full activity after 8-12 hour post-administration.

In vivo gene knockdown and biodistribution of dTAT complexes. The dTAT complexes were also investigated for gene silencing in vivo. The siRNA-induced gene silencing was determined by tail vein injection of dTAT complexed with GAPDH siRNA. The siRNA loading was maximized and these complexes were also condensed with calcium to yield a small particle size. Animals were given a 200 μL injection of either GAPDH siRNA (38.5 μM or 77 μM), control siRNA (77 μM), or PBS (Table 6). Tissues were analyzed 48 hours after administration.

TABLE 6

| Group | dTAT | siRNA | Glucose | CaCl₂ |
|---|---|---|---|---|
| number | (mg/mL) | (μM) | (%) | (mM) |
| 1 | 40 | 38.5 | 2 | 70 |
| 2 | 40 | 77 | 2 | 70 |
| 3 | 40 | 77 | 2 | 70 |
| 4 | (PBS control) | — | — | — |

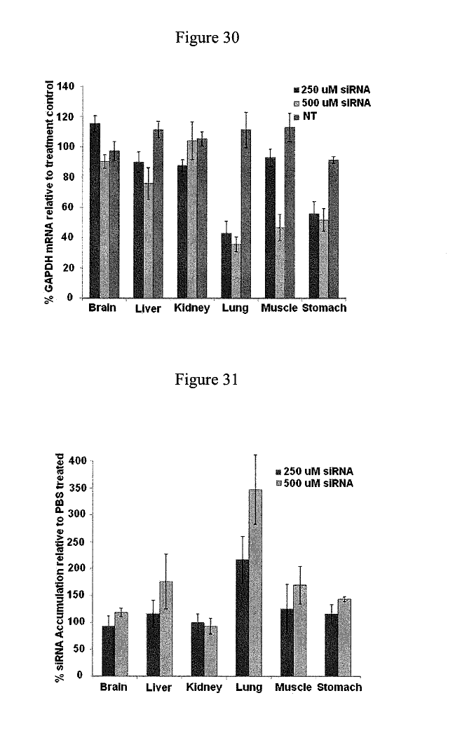

To assess the performance of GAPDH siRNA, the amount of GAPDH mRNA was determined in brain, liver, kidney, lung, muscle, and stomach. The relative knockdown was then determined by normalizing these data to the GAPDH mRNA expression in tissue samples from animals given PBS (FIG. 30). As expected, animals treated with control siRNA did not show GAPDH mRNA knockdown. At the higher GAPDH siRNA dose (77 μM), knockdown was most pronounced in lung, muscle, and stomach tissue. The lower siRNA dose (38.5 μM) also showed significant knockdown of GAPDH mRNA in lung tissue. Surprisingly, very little knockdown was noted in liver and kidney tissue.

The amount of GAPDH siRNA in these tissues was also quantified, which provided more direct evidence of the biodistribution of complexes. The amount of siRNA found in brain, liver, kidney, lung, muscle, and stomach was normalized to the amount of GAPDH siRNA in the corresponding tissues of animals receiving PBS (FIG. 31). This GAPDH siRNA is not expressed in mice, so all measured siRNA was due to delivery. GAPDH siRNA was detected at relatively high levels in lung tissue at both doses. The amount of siRNA in the lung tissue nearly doubled when the dose was doubled. The muscle and stomach tissue also showed significant siRNA accumulation at the higher dose (77 μM). Results were more variable in the liver tissue; however, some accumulation of GAPDH siRNA was evident at the higher dose.

Discussion

In this example, the effect of calcium on TAT and dTAT complexes was investigated to determine whether this formulation could effectively deliver siRNA while enhancing safety when compared to current vectors. Various N/P ratios of TAT and dTAT with different calcium concentrations and different doses of siRNA were used to optimize particle size and knockdown in vitro. Calcium was found to form compact TAT and dTAT complexes (58.5-201.3 nm) leading to high knockdown efficiencies in A549-luc-C8 lung epithelial cells. TAT and dTAT showed the smallest particle sizes at high calcium concentration (69.2 mM) across various N/P ratios. Small differences in the size of these complexes did not appear to directly affect gene silencing. A small siRNA dose of 10 nM was enough to knockdown luciferase expression by up to 87%. Also, it is important to note that absolutely no gene knockdown was observed for TAT and dTAT complexes that were not condensed with calcium. This provides support for the belief that calcium condensation is an essential part of the formulation.

The toxicity of TAT and dTAT was also systematically evaluated in vitro and in vivo. Low molecular weight CPPs were selected due to the potential for safe administration of these biodegradable polycations. Lung carcinoma cells studied here were highly tolerant of CPPs (IC₅₀≧4 mg/mL) and previous studies suggest that multiple other CPPs perform similarly (Baoum et al., 2009; Baoum and Berkland). The dose escalation of calcium condensed dTAT complexes administered to via tail vein injection confirmed the safety of this CPP. Even at exceptionally high doses of 1,000 mg/kg of dTAT, only a reversible behavioral phenotype was noted. In contrast, the LD₅₀ of PEI (25 kDa) was reported to be ˜4 mg/kg in mice. β-cyclodextrin-based polymers showed an LD₄₀ of 200 mg/kg with some compromise in gene delivery efficacy (Hwang et al., 2001). Results for the CPP complexes reported in this example suggested that dose-limiting toxicity may not be a bottleneck in the translation of this siRNA delivery system.

Knockdown of a target gene requires successful delivery of the siRNA to the tissue of interest; therefore, efficacy and biodistribution studies were undertaken. Studies confirmed both the delivery of GAPDH siRNA and the knockdown of the target in several tissues. The relative quantity of siRNA delivered to the tissue samples correlated moderately well to the knockdown in GAPDH mRNA. The knockdown was noticeable in multiple tissues despite the fact that siRNA doses were modest (˜19 and 38 μg/kg) in comparison to other reports. For example, doses of 150-250 μg/kg are common when using liposomal delivery systems (Landen et al., 2005). Researchers have dosed siRNA up to very high values, but are commonly limited by the toxicity of the carrier, when one is employed. In the case of CPP complexes shown in this example, the siRNA doses could be further increased, even without additional optimization of the current formulation approach. Thus, calcium condensed CPP complexes induced silencing efficacy in vivo and dose may be escalated to further improve performance.

Another difficulty with current siRNA delivery strategies is the accumulation of these colloids in organs of the reticuloendothelial system. Attempts to increase the dose of therapeutic to target tissue can be confounded by unwanted accrual in liver, kidney, spleen, or other organs. In this example, the low levels of siRNA in liver and kidney tissues of mice may provide an important advantage over existing delivery systems. One hypothesis for the preferential targeting of lung over liver tissue observed here is the evidence of small particle size for CPP complexes. CPP complexes condensed with calcium retained a very small size, which may have facilitated delivery to highly vascularized tissue such as the lung and muscle. In addition, formulation parameters may be adjusted to yield larger particles that may preferentially accrue in liver tissue if desired (Tables 4 & 5).

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A composition comprising:
a cationic polymer having a molecular weight of about 25,000 daltons or less;
a nucleic acid; and
a metal ion present in a range of about 5 to about 800 millimolar,
wherein the cationic polymer, the nucleic acid, and the metal ion are a complex having a size of about 500 nanometers or less.

2. The composition of claim 1 wherein the cationic polymer is selected from the group consisting of polyethylenimine, a cell penetrating peptide, and a cationic peptide.

3. The composition of claim 1 wherein the cationic polymer has a molecular weight of about 15,000 daltons or less.

4. The composition of claim 1 wherein the cationic polymer has a molecular weight of about 10,000 daltons or less.

5. The composition of claim 1 wherein the cationic polymer has a molecular weight of about 5,000 daltons or less.

6. The composition of claim 1 wherein the cationic polymer has a molecular weight of about 2,000 daltons or less.

7. The composition of claim 1 wherein the cationic polymer is a cell-penetrating peptide.

8. The composition of claim 1 wherein the cationic polymer is a HIV-1 TAT peptide.

9. The composition of claim 1 wherein the metal ion is calcium.

10. The composition of claim 1 wherein the complex has a size in the range of about 30 nanometers to about 150 nanometers.

11. The composition of claim 1 wherein the complex has a size less than or equal to about 150 nanometers.

12. The composition of claim 1 wherein the metal ion is present in a range from about 5 millimolar to about 500 millimolar.

13. The composition of claim 1 wherein the metal ion is present in a range from about 10 millimolar to about 250 millimolar.

14. The composition of claim 1 wherein the metal ion is present in a range from about 20 millimolar to about 150 millimolar.

15. The composition of claim 1 wherein the cationic polymer is a cationic peptide containing between about 30% and about 100% cationic amino acids.

16. The composition of claim 1 wherein the nucleic acid is RNA or DNA.

17. The composition of claim 1 wherein the nucleic acid is siRNA, shRNA, or miRNA.

18. The composition of claim 1 wherein the complex has an IC50 greater than 5 mg/ml.

19. The composition of claim 1 wherein the complex has an IC50 greater than 1 mg/ml.

20. The composition of claim 1 wherein the complex has an IC50 greater than 500 μg/ml.

21. A method comprising:
providing a composition comprising: a cationic polymer having a molecular weight of about 25,000 daltons or less, a nucleic acid, and a metal ion present in a range of about 5 to about 800 millimolar;
allowing the nucleic acid and the cationic polymer to form a polyplex having a size of about 250 nanometers or more; and
allowing the metal ion to condense the polyplex and form a complex having a size of about 500 nanometers or less.

22. The method of claim 21 wherein the cationic polymer has a molecular weight of about 15,000 daltons or less.

23. The method of claim 21 wherein the cationic polymer has a molecular weight of about 10,000 daltons or less.

24. The method of claim 21 wherein the cationic polymer has a molecular weight of about 5,000 daltons or less.

25. The method of claim 21 wherein the cationic polymer is a cell-penetrating peptide.

26. The method of claim 21 wherein the cationic polymer is a HIV-1 TAT peptide.

27. The method of claim 21 wherein the metal ion is calcium.

28. The method of claim 21 wherein the complex has a size in the range of about 30 nanometers to about 150 nanometers.

29. The method of claim 21 wherein the complex has a size less than or equal to about 150 nanometers.

30. The method of claim 21 wherein the cationic polymer is a cationic peptide containing between about 30% and about 100% cationic amino acids.

31. The method of claim 21 wherein the nucleic acid is RNA or DNA.

32. The method of claim 21 wherein the nucleic acid is siRNA, shRNA, or miRNA.

33. The method of claim 21 wherein the complex has an IC50 greater than 5 mg/ml.

34. The method of claim 21 wherein the complex has an IC50 greater than 1 mg/ml.

35. The method of claim 21 wherein the complex has an IC50 greater than 500 μg/ml.

36. A method comprising introducing into a tissue or a cell a composition comprising: a cationic polymer having a molecular weight of about 25,000 daltons or less; a nucleic acid; and a metal ion present in a range of about 5 to about 800 millimolar; wherein the cationic polymer, nucleic acid, and metal ion form a complex that is about 500 nm or less.
